(19) United States
(12) Patent Application Publication
(10) Pub. No.: US 2026/0253737 A1
(43) Pub. Date: Aug. 27, 2026

(54) System, Method, and Computer Readable Medium for Adaptive Bio-Behavioral Control (ABC) in Diabetes

(71) Applicant: University of Virginia Patent Foundation, Charlottesville, VA (US)

(72) Inventors: Boris P. Kovatchev, Charlottesville, VA (US); Patricio Colmegna, Zion Crossroads, VA (US); Jenny L. Diaz-Castañeda, Charlottesville, VA (US); Maria Fernanda Villa Tamayo, Charlottesville, VA (US)

(73) Assignee: University of Virginia Patent Foundation, Charlottesville, VA (US)

(21) Appl. No.: 19/159,204

(22) PCT Filed: Feb. 22, 2024
(86) PCT No.: PCT/US24/16960

§ 371 Date: Aug. 22, 2025

Related U.S. Application Data
(60) Provisional application No. 63/448,082, filed on Feb. 24, 2023.
(60) Provisional application No. 63/459,060, filed on Apr. 13, 2023.

(51) Int. Cl. G16H 50/30; G16H 10/60; G16H 20/17; G16H 20/60; G16H 50/50

(52) U.S. Cl. CPC G16H 50/30; G16H 10/60; G16H 20/17; G16H 20/60; G16H 50/50

(57) ABSTRACT

A method, system, and computer readable medium for implementing the Adaptive Bio-Behavioral Control concept, which allows for bi-directional, human-machine co-regulation of metabolic disorders, such as diabetes mellitus. The Adaptive Bio-Behavioral Control concept may encompass, but is not limited thereto, three modules: a) a Physiological Adaptation Module which processes a user's historical data to estimate a personalized model of the user's glucose metabolism and to calculate suggested in-sulin-dosing parameters; b) a Behavioral Adaptation Module which assists the user's adaptation to the treatment action by determining a plurality of actionable information and risk assessments based on the user's glucose monitoring records; and c) a Replay Module which computes a simulation of effects from hypothetical changes to the user's treatment or behavior based on a personalized model of the user's glucose metabolism.

### CROSS REFERENCE TO RELATED APPLICATIONS

The present application claims benefit of priority under 35 U.S.C § 119(e) from U.S. Provisional Application Ser. No. 63/448,082, filed Feb. 24, 2023, entitled “System, Method, and Computer Readable Medium for Adaptive Bio-Behavioral Control in Diabetes” and U.S. Provisional Application Ser. No. 63/459,060, filed Apr. 13, 2023, entitled “System, Method, and Computer Readable Medium for Adaptive Bio-Behavioral Control in Diabetes”; the disclosures of which are hereby incorporated by reference herein in their entirety.

### STATEMENT OF GOVERNMENT INTEREST

This invention was made with government support under DK085623 awarded by the National Institutes of Health. The government has certain rights in the invention.

### FIELD OF INVENTION

The present disclosure relates generally to medicine and medical devices, as used for insulin or medication treatment of diabetes mellitus and other metabolic disorders. More particularly, the present disclosure presents a method, system, and computer readable medium for implementing the Adaptive Bio-Behavioral Control concept, which improves human-machine co-regulation of metabolic disorders.

### BACKGROUND

Human-Machine Co-Adaptation-Necessity and Potential Solutions: A 2016 review underscored a well-known critical challenge to technological treatments of diabetes: “intra-individual variability in insulin requirements vary considerably within individuals with type 1 diabetes (TID) by on average 30% overnight and 20% during waking hours.” 1 This variation results from an underlying bio-behavioral process, driven by variation in human physiology such as intra- or inter-day changes in the rate of endogenous glucose production, insulin action and/or insulin sensitivity, all of which are time-dependent,²⁻⁵ and amplified by self-treatment behaviors, such as insulin mistiming or bolus/basal imbalance, missed meals or excessive exercise, which may trigger hypo- or hyperglycemia.^{6,7} However, while the need for an algorithm to adapt to changes in physiology is well recognized, it is less understood that in a Human-Machine system, the person would have to adapt to his/her mode of treatment as well.

Adaptation to Changes in Physiology: A landmark event that helped accelerate artificial pancreas (AP) research in humans without the need of preclinical animal data was the FDA endorsement of the UVA-Padova TID Simulator introduced by our team in 2008.⁸ The simulator was based on phenotypic data of carbohydrate metabolism obtained from studies conducted in >200 adults and children applying a triple tracer technique to derive key metabolic indices and parameters.⁹⁻¹⁶ This simulator has since been updated periodically based on emerging physiological data related to diurnal patterns of insulin sensitivity, dawn phenomenon, and glucagon regulation and action amongst others.²⁻⁵ Thus, quantitative models of physiological changes due to circadian factors and behavioral perturbations exist and are well within the realm of our past and current metabolic modeling efforts.¹⁷ In the past 10 years these models enabled countless in silico experiments assisting the development of control algorithms and the testing of algorithm adaptation to physiology, which is key to further improvement and automation of upcoming AP designs, e.g. removal of meal/exercise announcements.^{17,18} This said, real-time adaptation of a control algorithm to physiological changes is challenging, primarily due to the limited available inputs (e.g., continuous glucose monitoring (CGM) and possibly other signals). Nevertheless, a 12-week multi-site study (provided in part by the present inventor) involving 30 participants with TID and using AP algorithm parameter adaptation once a week, yielded significant reductions in HbA_{1c} and hypoglycemia.¹⁹ Further, long-term parameter adaptation introduced as a superstructure to an AP algorithm has shown promising in silico results,²⁰ reduced average glucose from 173 mg/dL to 138 mg/dL (p<0.001) and improved percent time in the target range 70-180 mg/dL from 64% to 93% (p<0.001), without additional risk of hypoglycemia. Thus, both clinical and in silico results indicate the feasibility of weekly algorithm parameter adaptation to changes in human physiology.²⁰

Behavioral Adaptation to the Action of AP. Recent data show that system problems and frustrations cause 38% of users to stop using AP in a real-world setting within 9 months.²¹ In addition to AP system issues, unrealistic expectations of the device and poor fit with the user's lifestyle all contribute to such an outcome. Thus, behavioral adaptation to technology should be considered when making decisions whether an AP device is fit-for-purpose in the context of lived experience. An early formal mathematical description of this human-machine interaction process was given by our Stochastic Model of Self-Regulation, which provided a probabilistic interpretation of the sequence: internal condition to perception/awareness to appraisal and to self-regulation decision.⁶ Following this work, our Bio-Psycho-Behavioral Model of severe hypoglycemia described the precursors and the warning signs of hypoglycemic episodes and identified points for intervention to reduce the risk.²²⁻²⁴ The approach was employed by the Blood Glucose Awareness Training (BGAT), an extensively used behavioral intervention in diabetes.²⁵⁻⁴⁷ Over the past few years, our group has combined Ecological Momentary Analysis (EMA) with CGM data to address clinically important questions regarding relationships between blood glucose (BG) levels and behavioral variables, such as the association between psychological stress and BG patterns using AP algorithms, where we found a small but significant association between stress and glycemic instability.⁴⁸ Another approach is KALMOD, based on the Kaleidoscope Model of Care, which identifies personal barriers to optimal self-management for non-adherence, providing specific and tailored advice to each individual according to their current situation to overcome those barriers.^{49,50} However, although the critical role that individual psycho-behavioral characteristics play in diabetes management and control is well-documented,⁵¹ little is known how such variables may relate to achieving the optimal benefits of AP. Using data from major AP clinical trials, we are investigating psychological and behavioral characteristics that may influence this process, which is the first step toward developing empirically-based patient education and intervention programs specifically designed to enhance likelihood of successful use and outcomes with technology.

### SUMMARY OF ASPECTS OF EMBODIMENTS OF THE PRESENT INVENTION

An aspect of an embodiment of the present invention generally relates to medicine and medical devices, as used for insulin or medication treatment of diabetes mellitus and other metabolic disorders, including but not limited to TID and type 2 diabetes (T2D), latent autoimmune diabetes in adults (LADA), postprandial or reactive hyperglycemia, or insulin resistance. In alternative embodiments, the invention tracks the physiological and behavioral characteristics of a person with diabetes, in relationship to the technology they use, and facilitates the user-system co-optimization of diabetes control. An advantage of an embodiment of the invention is, but not limited thereto, the Adaptive Bio-Behavioral Control (ABC) method, comprised of two modules: (1) A Physiological Adaptation Module (PAM) that processes a user's historical data to estimate a personalized model of the user's glucose metabolism and calculate suggested insulin-dosing parameters, and (2) A Behavioral Adaptation Module (BAM) that assists the person's adaptation to the treatment action by determining a plurality of actionable information and risk assessments based on the user's glucose monitoring records. In addition, the present invention introduces a Replay Module that may be used to compute a simulation of effects from hypothetical changes to the user's treatment or behavior based on said personalized model of the user's glucose metabolism.

The ABC method addresses human-machine co-regulation of metabolic disorders via several interacting cycles developing on different time scales: (1) First, the ABC process is initialized by individual characteristics, such as HbA1c, history of severe hypoglycemia, or hypoglycemia unawareness that set the stage for further control adaptation; this initialization is slow, develops over months or years; (2) Natural variation in a person's metabolic parameters occurs within a day (e.g. circadian rhythms) and between days, other changes are possible, e.g. due to illness; (3) Behavioral perturbations challenge the metabolic system with meals or physical activity, which may trigger low or high BG events and escalate to recurrent bypo- or hyperglycemia, respectively; this cycle includes significant randomness and is therefore best described by a stochastic process. This bio-behavioral interplay amplifies or attenuates the BG fluctuations of a person and is detected as degree of glucose variability by CGM and/or other signals. In this context, the objective of the ABC co-regulation method is two-fold: (1) Gradual and safe stabilization of the metabolic system by adapting control parameters to the specifics and physiological variation of the metabolic system, and (2) Fast response to biobehavioral challenges that may require behavioral adaptation by the user, e.g. around meals and exercise. In one embodiment, the ABC method consists of a web-based system for human-machine co-adaptation in diabetes. The system encompasses two main modules: (1) PAM which tracks metabolic changes and adapts the treatment parameters accordingly, and (2) BAM which assists the person's adaptation to the treatment action via information feedback.

Potential applications of the Method include: (i) Diabetes decision support systems, (ii) Computerized diabetes education allowing each person to replay their own treatment scenarios in computer simulation, (iii) Bi-directional system to user and user to system co-adaptation of automated insulin delivery (AID) systems, known as the “artificial pancreas”, (iv) Titration of insulin and/or other medications in newly diagnosed T2D, or (v) Tracking and in silico assessment of disease fluctuations and progression.

The ABC method can work with any CGM device and any closed-loop control (CLC) or continuous subcutaneous insulin delivery (CSII) system, in several modes of operation, but not limited thereto: (1) BAM only, providing a plurality of actionable information and risk assessments to the user; (2) PAM only, estimating a personalized model of the user's glucose metabolism and calculating insulin-dosing parameters, recommended to or automatically implemented by, an AID system, (3) BAM and PAM, providing bi-directional human-machine adaptation support, (4) PAM only and replay simulation capabilities, or (5) BAM, PAM and replay simulation capabilities.

An aspect of an embodiment of the present invention provides, among other things, a system, method, and computer readable medium for implementing the ABC method.

An aspect of an embodiment of the present invention provides, but not limited thereto, an application for human-machine co-adaptation in diabetes; such as for the treatment to adapt to the person and the person to adapt to the treatment. In an embodiment the application may be web-based among other approaches discussed herein. An aspect of an embodiment of the present invention provides, among other things, a system, method, and computer readable medium that encompasses, but not limited thereto, three modules: a) PAM which processes a user's historical data to estimate a personalized model of the user's glucose metabolism and to calculate suggested insulin-dosing parameters, b) BAM which assists the user's adaptation to the treatment action by determining a plurality of actionable information and risk assessments based on the user's glucose monitoring records, and c) a Replay Module which computes a simulation of effects from hypothetical changes to the user's treatment or behavior based on a personalized model of the user's glucose metabolism.

An aspect of an embodiment of the present invention provides, among other things, a processor-based method for improving human-machine co-regulation of metabolic disorders. The method comprising: receiving a user's historical data, the user's historical data including the user's glucose monitoring records and insulin therapy profile; determining a plurality of actionable information and risk assessments to assist the user's behavioral intervention based on user's glucose monitoring records; processing the historical data to estimate a personalized model of the user's glucose metabolism; determining insulin-dosing parameters based on the personalized model of the user's glucose metabolism; and computing a simulation of effects from hypothetical changes to the user's treatment or behavior based on the personalized model of the user's glucose metabolism.

An aspect of an embodiment of the present invention provides, among other things, a system for improving human-machine co-regulation of metabolic disorders. The system comprising: a computer processor and a memory configured to store instructions that are executable by the computer processor. The computer processor is configured to execute instructions for: receiving a user's historical data, the user's historical data including the user's glucose monitoring records and insulin therapy profile; determining a plurality of actionable information and risk assessments to assist the user's behavioral intervention based on user's glucose monitoring records; processing the historical data to estimate a personalized model of the user's glucose metabolism; determining insulin-dosing parameters based on the personalized model of the user's glucose metabolism; and computing a simulation of effects from hypothetical changes or non-changes to the user's treatment or behavior based on the personalized model of the user's glucose metabolism.

An aspect of an embodiment of the present invention provides, among other things, a non-transitory computer readable medium having program instructions for use in human-machine co-regulation of metabolic disorders. The execution of the program instructions by one or more processors of a computer system causes the one or more processors to: receive a user's historical data, the user's historical data including the user's glucose monitoring records and insulin therapy profile; determine a plurality of actionable information and risk assessments to assist the user's behavioral intervention based on user's glucose monitoring records; process the historical data to estimate a personalized model of the user's glucose metabolism; determine insulin-dosing parameters based on the personalized model of the user's glucose metabolism; and compute a simulation of effects from hypothetical changes to the user's treatment or behavior based on the personalized model of the user's glucose metabolism.

An aspect of an embodiment of the present invention provides, among other things, a method, system, and computer readable medium for implementing the Adaptive Bio-Behavioral Control concept, which allows for bi-directional, human-machine co-regulation of metabolic disorders, such as diabetes mellitus. The Adaptive Bio-Behavioral Control concept may encompass, but is not limited thereto, three modules: a) a Physiological Adaptation Module which processes a user's historical data to estimate a personalized model of the user's glucose metabolism and to calculate suggested insulin-dosing parameters, b) a Behavioral Adaptation Module which assists the user's adaptation to the treatment action by determining a plurality of actionable information and risk assessments based on the user's glucose monitoring records, and c) a Replay Module which computes a simulation of effects from hypothetical changes to the user's treatment or behavior based on a personalized model of the user's glucose metabolism.

The invention itself, together with further objects and attendant advantages, will best be understood by reference to the following detailed description, taken in conjunction with the accompanying drawings.

These and other objects, along with advantages and features of various aspects of embodiments of the invention disclosed herein, will be made more apparent from the description, drawings and claims that follow.

Additional descriptions of aspects of the present disclosure will now be provided with reference to the accompanying drawings. The drawings form a part hereof and show, by way of illustration, specific embodiments or examples.

### BRIEF DESCRIPTION OF THE DRAWINGS

The foregoing and other objects, features and advantages of the present invention, as well as the invention itself, will be more fully understood from the following description of preferred embodiments, when read together with the accompanying drawings.

The accompanying drawings, which are incorporated into and form a part of the instant specification, illustrate several aspects and embodiments of the present invention and, together with the description herein, serve to explain the principles of the invention. The drawings are provided only for the purpose of illustrating select embodiments of the invention and are not to be construed as limiting the invention.

FIG. 1 schematically illustrates an example of the ABC Concept.

FIG. 2 schematically illustrates an embodiment of the system architecture (and related method) of the web-based ABC system.

FIG. 3 schematically illustrates an example of the integration of PAM into the ABC cycle.

FIG. 4 schematically illustrates an example of the integration of BAM into the ABC cycle.

FIG. 5 schematically illustrates an example of the combination of BAM and PAM modules to define the adaptive bio-behavioral treatment.

FIG. 6 schematically illustrates an example of the user interface (UI) flow diagram of the Web Information Tool (WIT).

FIG. 7 schematically illustrates an example of the web implementation of BAM-Entry and Status Overview.

FIG. 8 schematically illustrates an example of the web implementation of BAM-Risk Information.

FIG. 9 schematically illustrates an example of the web implementation of BAM-commonly accepted glucose charts (CAGC).

FIG. 10 schematically illustrates an example of the web implementation of PAM-Architecture.

FIG. 11 schematically illustrates an example of the web implementation of the In Silico Replay Module.

FIG. 12 is a block diagram illustrating an example of a machine upon which one or more aspects of embodiments of the present invention can be implemented.

### DETAILED DESCRIPTION OF EXEMPLARY EMBODIMENTS

### Defining the ABC Process

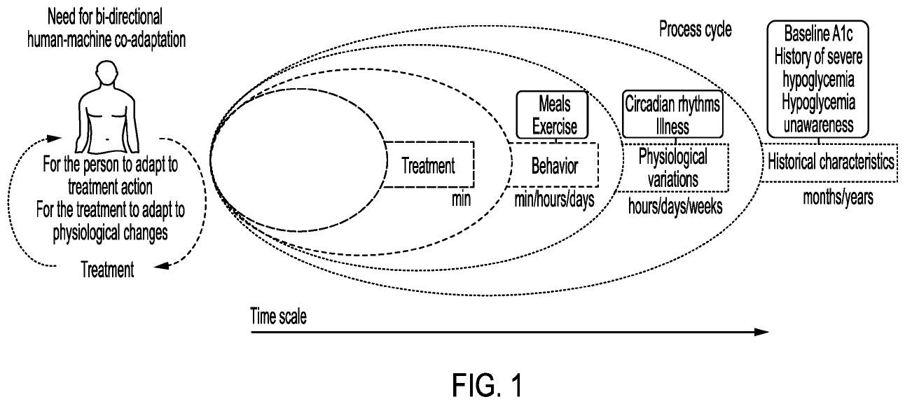

Referring to FIG. 1, the ABC process consists of interacting cycles developing on different time scales:⁵² (1) First, it is initialized by individual characteristics, such as HbA1c, history of severe hypoglycemia, or hypoglycemia unawareness that set the stage for further control adaptation; this initialization is slow, develops over months or years, and can be favorable or unfavorable to AP (e.g. individuals at overall good vs. poor control would fare differently using AP;⁵³⁻⁵⁵ (2) natural variation in a person's metabolic parameters occurs within a day (e.g. circadian rhythms) and between days; slower changes are possible, e.g. due to illness; and (3) behavioral perturbations challenge the metabolic system with meals or physical activity, which may trigger low or high BG events and escalate to recurrent hypo- or hyperglycemia, respectively; this cycle includes significant randomness and is therefore best described by a stochastic process. This bio-behavioral interplay amplifies or attenuates the BG fluctuations of a person and is detected as degree of glucose variability by CGM and/or other signals. In this context, the control co-regulation objective is two-fold: gradual and safe stabilization of the metabolic system by adapting control parameters to the specifics and physiological variation of the metabolic system, and fast response to biobehavioral challenges that may require behavioral adaptation by the user, e.g. around meals and exercise.

The overall objective of the ABC strategy is to enable a new generation of technologies which will: (i) provide information to the user to inform user's adaptation to the technological treatment system and (ii) automatically adapt the action of the control algorithm to follow daily changes in physiology and behavior. To the best of our knowledge, such bi-directional human-machine co-adaptation has not been attempted before, partially due to underestimation of the behavioral challenges related to AP use, and partially due to the relative immaturity of AP systems, which until recently could not sustain long-term use without facing significant technical problems or requiring extensive user involvement.

### ABC System Architecture

In this section, a general description of the ABC system is presented in terms of its functionality, and how its components interact with each other using CGM and insulin pump data, the system can characterize glycemic control, optimize insulin therapy profiles, and simulate changes to treatment decisions. All computation is performed in the background, i.e., without requiring user intervention. Through a simple and intuitive interface, users can: (i) track glucose metrics, (ii) obtain suggestions for their insulin treatment parameters, and (iii) simulate changes to basal and bolus settings. Although this application can be used by health-care providers, the main objective of this software is to empower patients to manage their own diabetes through an educational and decision-support tool.

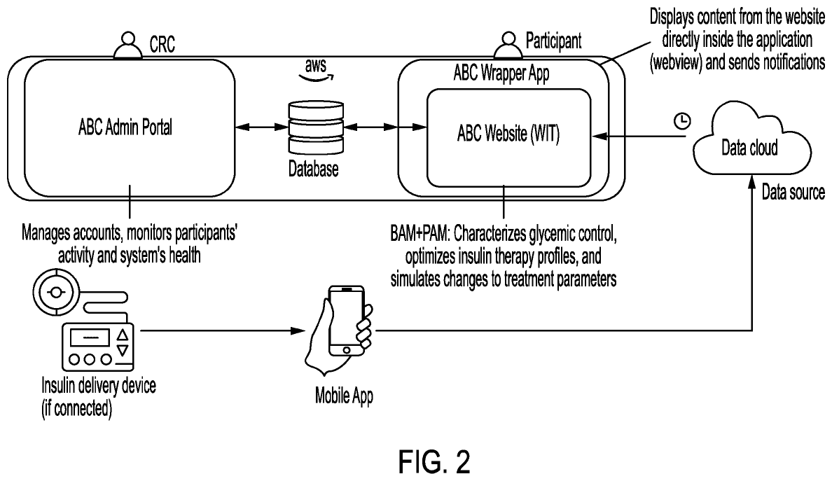

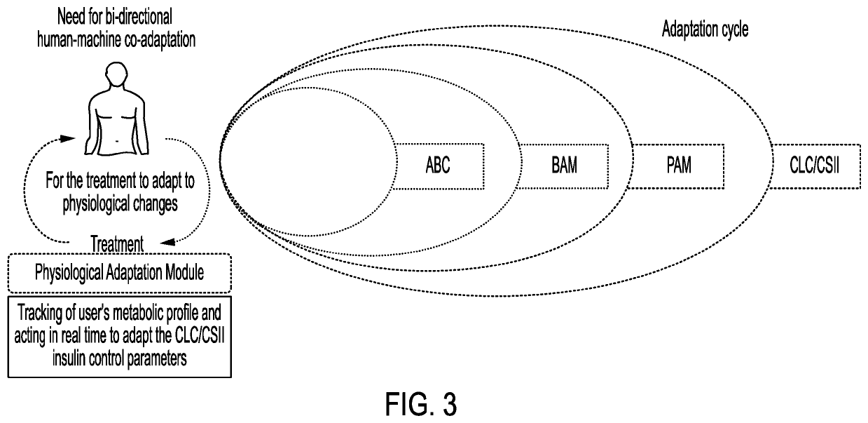

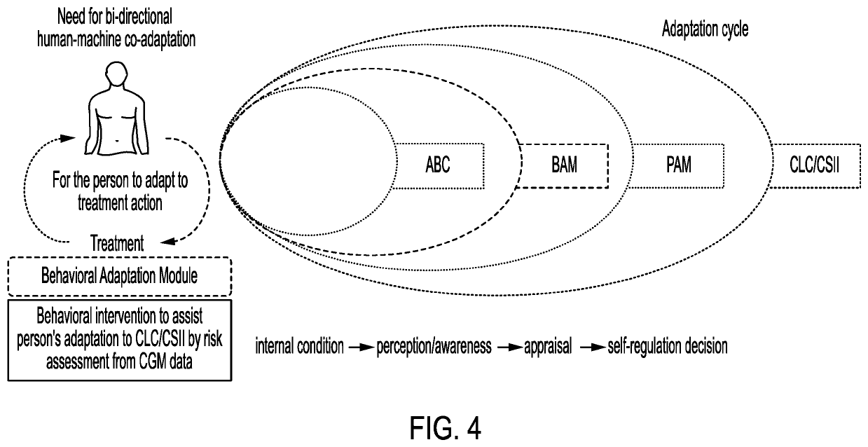

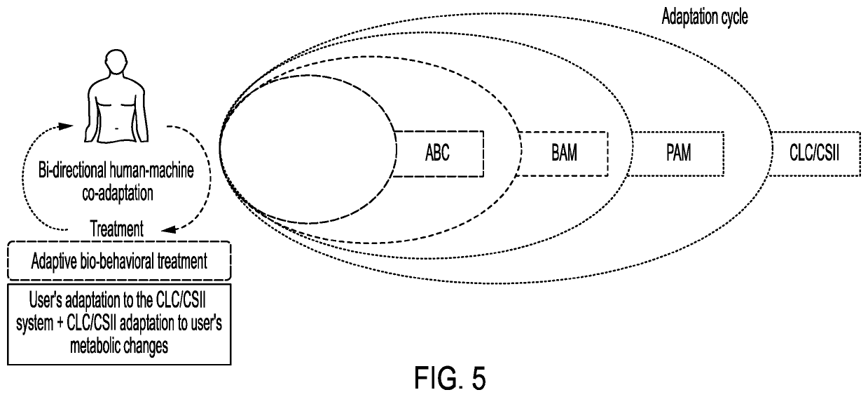

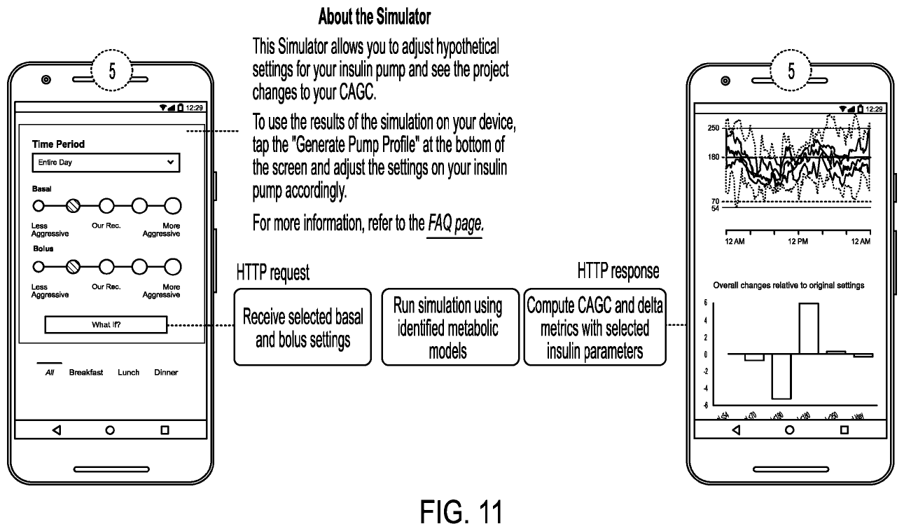

An embodiment of the web-based ABC system architecture is presented in FIG. 2. The ABC system application consists of a number of subsystems:
The ABC Admin Portal manages accounts, monitors users' activity and system's health that may include a clinic research coordinator (CRC), e.g., a monitor;
Database Layer: An Amazon Relational Database (RD) that stores field-collected data from users' insulin pumps and module-specific variables for system operation, including but not limited to account profiles, metabolic models and optimization results. SQLAlchemy can be used to interact with the database;
The ABC Website (WIT) contains the ABC system modules BAM, PAM, and the Replay Module, which characterize glycemic control, optimize insulin therapy profiles, and simulate changes to treatment parameters:
   The Physiological Adaptation Module (PAM); Processes the user's historical data to estimate a personalized model of the user's glucose metabolism, and to determine insulin-dosing parameters. PAM estimates the personalized model of the user's glucose metabolism on a daily basis, and optimizes the Basal Rate (BR), Carbohydrate Ratio (CR), and Correction Factor (CF) profiles on a biweekly basis (Auto-titration Module (ATM)). The function of the PAM within the context of ABC, is presented in FIG. 3. This module is typically associated with insulin delivery parameter adaptation that uses CLC or CSII.
   Behavioral Adaptation Module (BAM): Determines a plurality of actionable information and risk assessments to assist the user's behavioral intervention based on user's glucose monitoring records. For example, BAM computes time spent in different glucose ranges, estimates the estimate mean glucose (EMG), calculates risks of hypo- and hyperglycemia, and generates commonly accepted glucose charts (CAGC). The function of the BAM within the context of ABC is presented in FIG. 4. This module is typically associated with advanced technologies, e.g., CLC, CSII, Insulin Pens, that require user's adaptation to their functioning. In an embodiment, glucose management indicator (GMI) may be implemented instead of estimate mean glucose (EMG). In an embodiment, ambulatory glucose profile (AGP) graphs may be implemented instead of commonly accepted glucose charts (CAGC).
   In Silico Replay Module: Computes a simulation of effects from hypothetical changes to the user's treatment or behavior based on personalized model(s) of the user's glucose metabolism. The Replay Module, thereby, allows users to simulate changes to treatment parameters, such as the recommended basal and bolus settings. For example, users can send simulation requests to the system, asking, for instance, to simulate the impact of lowering their BR over a certain period of time. The system receives the request, queries data from the database, runs the simulation, and sends the results back to the UI, where they are displayed to the user. An embodiment of the web implementation of the In Silico Replay Module is presented in FIG. 11.
The Wrapper App (WAPP FIG. 2); Includes a UI to display content from the website directly inside the application and send notifications to drive users' attention to important elements when needed. The WAPP may be used to implement the entire ABC process and drives bi-directional human-machine co-adaptation (FIG. 5).

As depicted in FIG. 2, the system does not interface directly with the user's insulin pump. A third-party system (typically implemented as a mobile app) connected to a network (typically the Internet) uploads data from the user's insulin pump to a dedicated server automatically on a regular basis.

### Core Operation:

WIT uses asynchronous processing to handle the UI and synchronous processing for periodic tasks such as data collection, EMG computation, therapy optimization and virtual image generation.
Synchronous processing
   About every two hours,
      a WIT fetches backend files from device-related Cloud repository, stores them in a local repository, and keeps records of disconnections/re-connections between the system devices.
      If a disconnection is detected, WIT sends an email notification to the monitor and WAPP pushes a notification to the user. If the disconnection persists, a new reminder is sent every 2 hours during the day and every 4 hours during the night. When connection is regained, another notification is generated. Users can enable a night mode to silence notifications between midnight and 6 am,
   Every day,
      WIT fetches data from the repository, feeds processed data into the Virtual Image Generator, and saves the results in the system's database (DB).
      WAPP sends a BAM notification to the users to encourage them to check their data. The notification time can be adjusted by the users to suit their daily schedule.
   Every two weeks,
      WIT fetches data from the system's DB, feeds data into an ATM, modulates BR and CR/CF profiles applying changes to each X-hour time segments, obtains the optimal modulation, saves the results in the system's DB and sends an email notification to the system monitoring staff. The length of the time segment X can be 2, 4, 6, 12, 24, or any number of hours.
      WAPP sends a PAM notification to the users in case a new suggestion for their pump settings is available on the system.
Asynchronous processing: Upon request by user, WIT triggers either the BAM module or the PAM module.
   BAM module fetches data from the repository and server memory, runs data processing, calls the corresponding method(s), and updates data displayed on the screen.
   PAM module levels insulin treatment profiles to have up to several time segments and implementable parameter values, fetches data from the system's DB and user interface, displays suggestions, executes simulations, and updates data displayed on the screen. The number of time segments can vary, depending on the capability of the CLC or CSII system used.

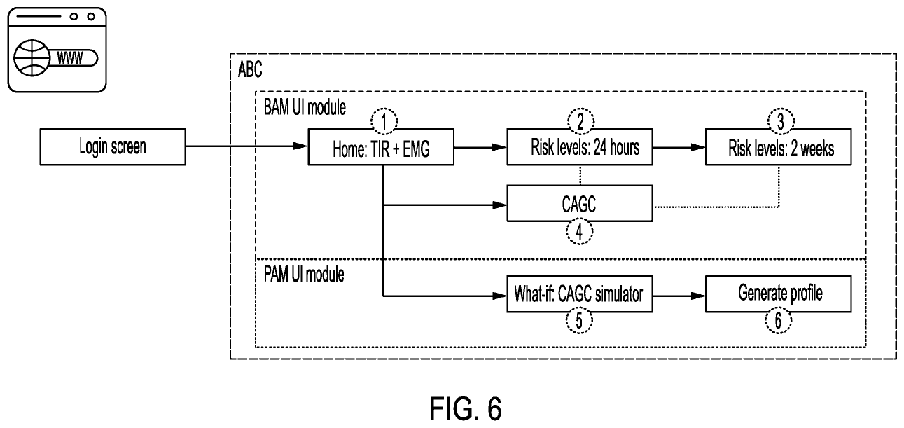

FIG. 6 shows the WIT flowchart. In an embodiment, WIT is implemented as a Flask application that generates the dynamic content, such as simulated data or glycemic metrics. Nginx is the front facing web server that handles the Hypertext Transfer Protocol (HTTP) requests that come in from the Internet, proving the static content (css, javascript, etc.) back to the requester. Gunicorn is a Web Server Gateway Interface (WSGI) HTTP server that lives between Nginx and Flask, i.e., it receives requests from Nginx and routes them to the Flask application. To handle multiple concurrent HTTP requests, Gunicorn is configured to use a mix of workers and threads.

The ABC system and its WIT can be implemented in any other software environment that is capable of supporting its core functions, such as described in the next section.

### ABC System Implementation

### Physiological Adaptation Module (PAM)

Every day, data are processed for the Virtual Image Generator to estimate a personalized model of the user's glucose metabolism. Every two weeks, all identified models are used by the ATM in an optimization procedure where the BR, CR, and CF profiles are modulated so as to minimize a cost function that weights the risk of hypoglycemia (low blood glucose index (LBGI)), risk of hyperglycemia (high blood glucose index (HBGI)) and the number of estimated hypo-treatments per day (number of times rescue carbohydrates are provided). The process flow can be summarized as follows:
1. Every day:
   i. The Data Fetching, Parsing and Pre-Processing modules are executed to capture and process CGM, insulin, therapy profile, meal, and controller-specific records.
   ii. Preprocessed data are parsed into “extended days”: each extended day is obtained from a 24-hour calendar day by adding a 6-hour head and a 2-hour tail.
   iii. A logistic regression-based detection algorithm is executed using insulin and CGM records to reconstruct and amend the meal record of the user. The method calculates the value of numerous features for each 5-minute interval of the day and based on the combination of those features and their respective coefficients determines if a meal is likely to have occurred at that time.
   iv. Each extended day is marked to be valid if:
      a. CGM gaps are no longer than 3 hours.
      b. At least 70% of CGM data are available.
      c. At least 2 boluses are recorded.
   v. For each valid extended day, the following steps are performed:
      a. A patient-specific mathematical model of glucose-insulin interaction in presence of meal ingestion and subcutaneous insulin administration is identified on the available data.
      b. Based on the identified model, an additive net-effect signal is estimated through model inversion by deconvolution to capture unmodeled uncertainty and closely reconstruct the available CGM data.
   vi. Results including processed data, model parameters and net-effect signal are stored in the system's DB.
2. Every two weeks:
   i. Last several days (e.g., 14 days or 7 days), are replayed using the personalized metabolic models combined in a closed-loop fashion with the AID system—for instance, Control-IQ. During this process, replayed days associated with a root mean square error (RMSE) greater than a certain predefined threshold (e.g., 25 mg/dL) are dropped and will not be considered in the optimization process.
   ii. Given the subset of valid days, an optimization process is performed to obtain optimal modulation factors for the BR, CR, and CF profiles. This process consists of resimulating the valid days, each time modulating the profiles differently so as to minimize the associated cost function.

### Optimal Insulin-Dosing Parameters

Biweekly, insulin-dosing parameters (BR, CR and CF) are determined jointly by solving an optimization problem that minimizes the weighted sum of the risks of hypoglycemia (LBGI) and hyperglycemia (HBGI) and the number of hypoglycemic treatments per day, as shown below:

$${min}_{\{ {α}_{1} , … , {α}_{6} \} \{ {β}_{1} , … , {β}_{6} \}} J = f ( {BR}^{′} , {CR}^{′} , {CF}^{′} )$$

$$subject to$$

$${α}_{i} ∈ [ - 1 , 1 ] ∀ i = 1 , … , 6$$

$${β}_{i} ∈ [ - 1 , 1 ] ∀ i = 1 , … , 6$$

with α^{v}={α₁, . . . , α₆} and β^{v}={β₁, . . . , β₆} the vector of modulation factors for CR-CF and BR in segments with predefined lengths (e.g. 2, 4, 6, 8, 12, 24 hours), respectively, and BR′, CR′, and CF′, the modulated profiles through the day for BR and CR-CF computed as a function of α^{v} and β^{v} as follows:

$${BR}^{′} = {BR}_{nom}^{v} \cdot ( 1 + P * \frac{{{β}_{ext}}^{v}}{100} )$$

$${CR}^{′} = {CR}_{nom}^{v} \cdot ( 1 + P * \frac{{α}_{ext}^{v}}{100} )$$

$${CF}^{′} = {CF}_{nom}^{v} \cdot ( 1 + P * \frac{{α}_{ext}^{v}}{100} )$$

with

$${BR}_{nom}^{v} ,$$

$${CR}_{nom}^{v}$$

$$and$$

$${CF}_{nom}^{v}$$

the current profiles through the day for BR, CR, and CF in five-minute intervals, and

$${α}_{ext}^{v}$$

and βₑₓₜ^{v} the extended version of α^{v} and β^{v} through the day in five-minute intervals. Note that, based on the feasible domains for αᵢ and βᵢ and the equations to compute BR′, CR′, and CF′, the maximum allowable change between biweekly recommendations is limited to P percent with respect to the previous insulin-dosing parameters, and both CR and CF are modified in the same way by using the same modulation factor (αᵢ).
The cost function (J) is given by:

$$J = {\sum }_{j = 1}^{{N}_{days}} 2 \cdot {LBGI}_{j} + {HBGI}_{j} + {N}_{j , HT}$$

with N_{days} the number of days considered for the optimization (≤14 days), N_{HT} the number of hypoglycemic treatments per day, and LBGI and HBGI the hypoglycemia and hyperglycemia indices per day, computed as:

$${Risk}_{k} = {log BG}_{k}^{1.084} - 5.381$$

$${LBGI}_{j} = \begin{matrix}{\sum }_{k = 1}^{{N}_{samples}} 22.77 * {Risk}_{k}^{2} & ∀ {Risk}_{k} \leq 0 and {BG}_{k} < 70\end{matrix}$$

$${HBGI}_{j} = \begin{matrix}{\sum }_{k = 1}^{{N}_{samples}} 22.77 * {Risk}_{k}^{2} & ∀ {Risk}_{k} > 0 and {BG}_{k} > 180\end{matrix}$$

with Nₛₐₘₚₗₑₛ=288 the number of samples per day considering a sampling time equal to five minutes.

The optimization problem is solved in Matlab® by using the function( ) function and the interior-point optimization algorithm. Stopping criteria as maximum number of iterations and termination tolerance on the function value can be defined in order to reduce computational burden.

### Variable List

| | | | |
|---|---|---|---|
| Name | Description | Units | Type |

INPUTS

| | | | |
|---|---|---|---|
| DataDay | Cell array containing the | Multiple | 1-dimensional array of 17 days |
| | data and ident | | |
| | dictionaries for each | | |
| | extended day | | |
| DataDay.matIdent | Extended Matrix | None | (n + m) × (n + 1) matrix, where n is the |
| | containing the model for | | number of states in the extended |
| | the i-th extended day | | day, m is the number of inputs |
| DataDay.NE | Net effect signal for the | mg/dl/min | 1-dimensional array of 288 doubles |
| | i-th day | | |
| DataDay.Istates | Initial condition for the | Multiple | 1-dimensional array of n doubles |
| | state variables for the i- | | |
| | th day | | |
| DataDay.op | Vector containing the | Multiple | 1-dimensional array of n + 2 doubles |
| | operating point of the i- | | |
| | th day | | |
| DataDay. signals | Matrix containing the | Multiple | A 22 × 288 matrix of doubles |
| | signals for the i-th day | | |
| | including: cgm, basal, | | |
| | correction boluses, meal | | |
| | boluses, bolus, BR | | |
| | profile, CR profile, CF | | |
| | profile, announced | | |
| | meals, flag fBolex, flag | | |
| | delay bolus, target meal | | |
| | bolus calculator, flag | | |
| | automatic bolus, flag to | | |
| | overrode the dose, mode | | |
| | of controller, flag to | | |
| | decline the correction | | |
| | bolus, flag extended | | |
| | bolus, percentage of dose | | |
| | of the extended bolus, | | |
| | duration of the extended | | |
| | bolus, target for CIQ, | | |
| | flag for sleep mode, flag | | |
| | for exercise mode | | |
| DataDay.mealsData | Matrix containing the | Multiple | A 2 × 384 matrix of doubles |
| | meal data for the i-th | | |
| | extended day including: | | |
| | carbohydrates amount | | |
| | and meal index | | |
| | classification | | |
| DataDay.demographics | Vector containing | Multiple | 1-dimensional array of 4 doubles |
| | subject's demographic | | |
| | information: total daily | | |
| | insulin, body weight, | | |
| | height, and age | | |
| Params | Setup parameters for the | None | Dictionary storing the parameters |
| | replay function | | |
| Params.Flag_Opt | Flag indicating if the | None | integer |
| | replay function is used to | | |
| | obtain the baseline cgm | | |
| | trace or the resimulation | | |
| | obtained when changing | | |
| | the insulin-dosing | | |
| | profiles | | |
| Params.valid_day | Vector containing the | None | 1-dimensional array of integers |
| | valid days to optimize in | | |
| | case of resimulation | | |
| Params.day_ini | Initial day of the cycle | None | Int |

OUTPUTS

| | | | |
|---|---|---|---|
| Alpha_matrix | Matrix containing the | None | A 2 × 6 matrix of doubles |
| | optimal modulation | | |
| | factors for the BR and | | |
| | CR-CF profiles | | |
| optGluc | Matrix containing the | mg/dl | A 5 × 288 matrix of doubles |
| | 5^{th}, 25^{th}, 50^{th}, 75^{th}, and | | |
| | 95^{th} percentiles of the | | |
| | replayed cgm traces | | |
| | across the 14 days | | |
| optDiff | Vector containing the | % | A 1 × 6 matrix of doubles |
| | differences in glycemic | | |
| | metrics between | | |
| | replayed cgm and | | |
| | resimulated cgm using | | |
| | optimal profiles for BR | | |
| | and CR-CF | | |

### Behavioral Adaptation Module (BAM)

### General Description

The BAM aims to provide a behavioral intervention to assist a person's adaptation to the CLC system through the presentation of actionable information and risk assessments.

The five distinct pieces of information provided by BAM are divided into two sets:
1. Set 1
   a. Time spent in different glucose ranges;
   b. An EMG to assist in tracking average glycemia and long-term goal setting;
   c. A glucose variability index, which tracks daily glucose variation to assist with adjustments of meal insulin dosing or physical activity/exercise;
   d. Glucose median and percentiles across days.
2. Set 2
   a. Hypo- and hyperglycemia risk indices, which split the overall glucose variability into risks specific to hypoglycemia or hyperglycemia.

### Set 1

Time spent in different glucose ranges, glucose variability index (coefficient of variation: standard deviation/mean) and glucose median and percentiles are computed when there is at least 70% of CGM records for the time period of interest.

### EMG

Every night, the EMG is computed using the last 14 days of CGM data (from 12 AM to 12 AM each day) that are obtained from the Data Parsing Module. In presence of at least 70% of CGM data, the EMG value for the current (1-th) day is computed as:

$$EMG ( \% ) = 3.31 + 0.02392 \times [ mean glucose in mg / dl ]$$

If not enough CGM data are available, the process is skipped until the next day. In addition to the EMG value, the trend of EMG change over the previous seven days (current day included) is estimated through linear regression and mapped into a specific “arrow slope”. The slope of the arrow representing the EMG rate of change (emgROC), which will be displayed to the user, is computed based on the following lookup table and relevant thresholds for emgROC:

| | | emgROC Non- |
|---|---|---|
| emgROC | Arrow Slope | limiting Examples |
| <=a, | Rapid decrease | a could be −1.48 |
| where a < −1.0 mg/dL/min | (code: −2) | |
| >a AND <=b, | Moderate | a could be −1.48 |
| where a < −1.0 mg/dL/min | decrease | b could be −0.58 |
| where b is between −1.0 and −0.25 mg/dL/min | (code: −1) | |
| >b AND <=c, | No relevant | b could be −0.58 |
| where b is between −1.0 and −0.25 mg/dL/min | change | c could be 0.64 |
| where c is between 0.25 and 1.0 mg/dL/min | (code: 0) | |
| >c AND <=d, | Moderate | c could be 0.64 |
| where c is between 0.25 and 1.0 mg/dL/min | increase | d could be 1.64 |
| where d > 1 mg/dL/min | (code: +1) | |
| >=d, | Rapid increase | d could be 1.64 |
| where d > 1.0 mg/dL/min. | (code: +2) | |

### Set 2

### Hypo- and Hyperglycemia Risks

The module is written with modularity in mind so that each piece of information is computed separately and in a stand-alone manner. The module takes historical CGM data as input and processes it using the process_CGM_data( ) function. The processed CGM data is then used as input to the LBGI( ) and HBGI( ) functions. These functions compute the LBGI and HBGI values for each day of data passed in. The risk_indices( ) function returns the LBGI and HBGI values of the 24 hours prior to 06:00 of the current date as the hypoglycemia and hyperglycemia risk indices respectively.

The output of risk_indices( ) is passed to the ST_scale( ) function which converts these values to a common scale.

A single function named BAM( ) wraps the above functionality, and this wrapper function returns the results as a single Python dictionary.

### Variable List

| | | | |
|---|---|---|---|
| Name | Description | Units | Type |

INPUTS

| | | | |
|---|---|---|---|
| current_date | The current date as a string | | Python string |
| | with format | | |
| | ‘MM/DD/YYYY’ | | |
| timestamps | A list of timestamp strings | | Python list of |
| | with format | | strings |
| | ‘MM/DD/YYYY | | |
| | HH:MM:SS’ | | |
| BG_values | A list of blood glucose | mg/dl | Python list of floats |
| | values | | |
| BAMp | A dictionary of | | Python dictionary |
| | configuration values for the | | |
| | BAM | | |

OUTPUTS

| | | |
|---|---|---|
| BAM_dict | A Python dictionary with | Python dictionary |
| | entries for each of the four | |
| | pieces of information | |
| | provided by the BAM | |

### Processing of Time Series Data

A list of timestamps and a list of blood glucose values measured in mg/dL is passed into the module. The list of timestamps and associated blood glucose values are transformed into a Pandas dataframe which enables:
1. Sensor error values to be removed,
2. Intervals of length 24 hours (06:00 on day n to 06:00 on day n+1) with less than 110 data points to be removed,
3. A check to make sure that there is enough data from the most recent interval of length 24 hours, and
4. A check to make sure there is data from at least 8 intervals of length 24 hours.

All of these are accomplished via the process_CGM_data( ) function.

### ST Scale

A single function (ST_scale ( ) is used to convert the variability index, risk indices, and risk trace values to a common scale, the ST scale, with domain [0, 11]. The function takes a list of values to convert and a tuple of bounds (lower_bound, upper_bound) for the values in their current scale as input. The lower bound is mapped to 0 while the upper bound is mapped to 11. Any values passed in which are less than the lower bound are clamped to the lower bound, while any values passed in which are greater than the upper bound are clamped to the upper bound.

### Risk Indices

The two risk indices, the LBGI and HBGI, are computed using the following equations:

$$LBGI = \frac{\sum {r}_{l} ( {x}_{i} )}{n}$$

where rₗ(xᵢ)=22.77f(xᵢ)² if f(xᵢ)≤0, and 0 otherwise;

$$HBGI = \frac{\sum {r}_{h} ( {x}_{i} )}{n}$$

where rₕ(x)=22.77f(xᵢ)² if f(xᵢ)>0, and 0 otherwise; and where

$$f ( {x}_{i} ) = ( {ln ( {x}_{i} )}^{1.804} - 5.381$$

for blood glucose readings x₁, . . . , xₙ measured in mg/dL. In our case, the blood glucose readings come from the 24 hours prior to 06:00 of the current date.

### In Silico Replay Module

The system's UI allows users to simulate changes to the recommended basal and bolus settings. Changes can be applied to predefined time segments (e.g., 2,4,6,8,12, 24 hours). The data flow of this process is illustrated in FIG. 6. Users can send simulation requests to the system, asking, for instance, to simulate the impact of lowering their BR over a certain period of time. The system receives the request, queries data from the database, runs the simulation, and sends the results back to the UI, where they are displayed to the user.

### Variable List

| | | | |
|---|---|---|---|
| Name | Description | Units | Type |

INPUTS

| | | | |
|---|---|---|---|
| request | Structure that stores data | — | Python dictionary |
| | from UI, including selected | | |
| | time interval, basal and | | |
| | bolus modulators | | |

OUTPUTS

| | | | |
|---|---|---|---|
| res | Dictionary with the | — | JSON response |
| | following fields: | | |
| | quantiles: Simulated | | |
| | glucose median and | | |
| | percentiles, | | |
| | tirDiffs: Delta time in | | |
| | ranges and number of | | |
| | hypo-treatments per day | | |
| | between simulation and | | |
| | original data | | |

### Process Flow

Users can ask the system to run a simulation by tapping a button on the UI (e.g., see next section). This request is received by the server, which performs the following tasks to send back a response.

### Data Extraction

The first step is to parse the data received from the UI. Specifically, the system extracts the selected time range (e.g., entire day, 12 am-6 am, 6 am-12 pm, 12 pm-6 pm, 6 pm-12 am) and basal and bolus modulators (e.g., −20%, −10%, 0%, 10%, 20%) to build the modulation factors (pFactors).

The system's DB is queried to build the input for the Replay Module and the optimal modulation factors are combined with pFactors to define the final modulation profiles for BR, CR and CF. The Replay Module is run and its response (res) is then sent to the UI.

### Graphical User Interface (UI)

In one embodiment, the flow chart of the Graphical UI of the ABC system is presented in FIG. 6. This is now used in a large-scale clinical trial testing the ABC system. Other embodiments can be designed to accommodate the functions, such as described in the section above.

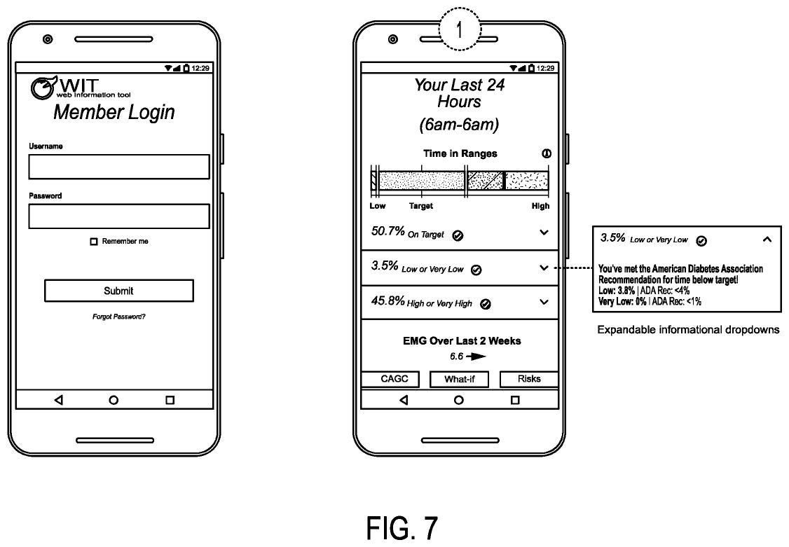

The first page that users see is the Login screen (FIG. 7) where they can gain access to the system by entering their username and password. The system offers a “Remember me” function to retain login details until users do a logout, and a “Forgot password?” function to allow users to reset their passwords using a reset token sent via email. Once logged in, users will be redirected to the home screen which time in range and EMG are displayed as part of BAM. Below the TIR graph, there are expandable informational dropdowns. At a glance, these tell users via a green or gray checkmark whether they have achieved their TIR goal in a particular range. If users want more information, they can expand the accordions and learn more without leaving the home screen. At the top of each screen of the web-based application, there is a connection status message that indicates whether the user's pump is properly connected to the mobile application that sends data to the cloud repository. Also, some screens contain tooltips that users can tap to learn more about the system or diabetes care.

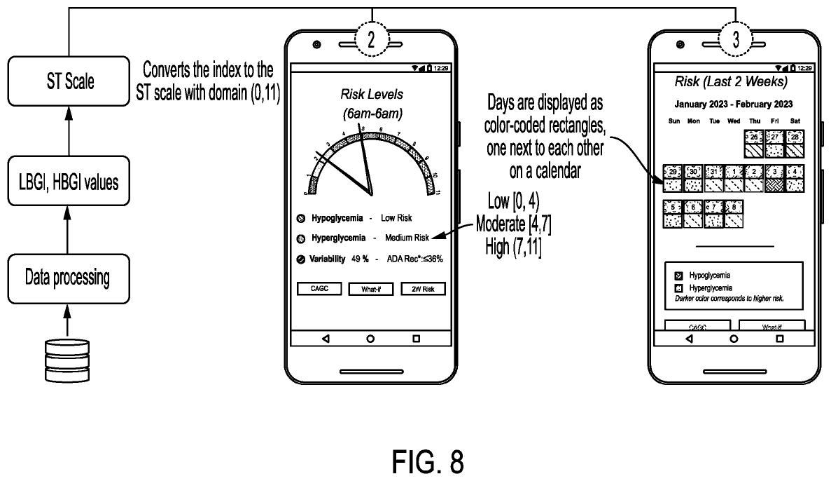

The next two screens of the BAM are about hypo- and hyperglycemia (FIG. 8). LBGI and HBGI values for the last 24 hours are scaled from 0 to 11, and values are reflected on a gauge with two needles: one for hypoglycemia and one for hyperglycemia. Below the gauge, risks are classified as low, moderate or high and glucose variability is also reported. Risks for the last 2 weeks are displayed on a calendar, where each day contains two color-coded rectangles with darker color corresponding to higher risk.

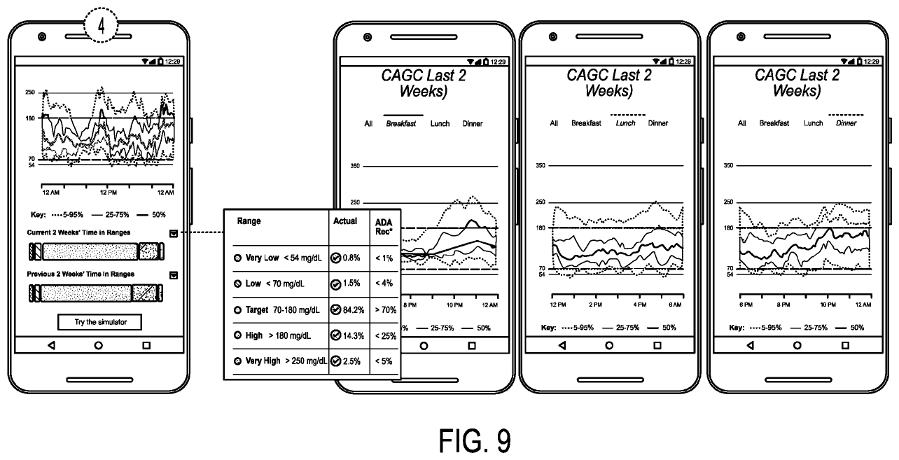

Further screens of the BAM present (CAGC), where users can see the traditional envelope graph with median and percentiles, and below it they can see the TIR bars for the current 2-week period and the previous 2-week period in order to track changes (FIG. 9). There is a tabbed interface where users can see (CAGC) from midnight to midnight or for different 6-hour time segments. The TIR bars are updated accordingly. At the bottom of the screen, users can get access to the PAM, which is the CAGC simulator and provides the personalized model of the user's glucose metabolism and original insulin-dosing parameters.

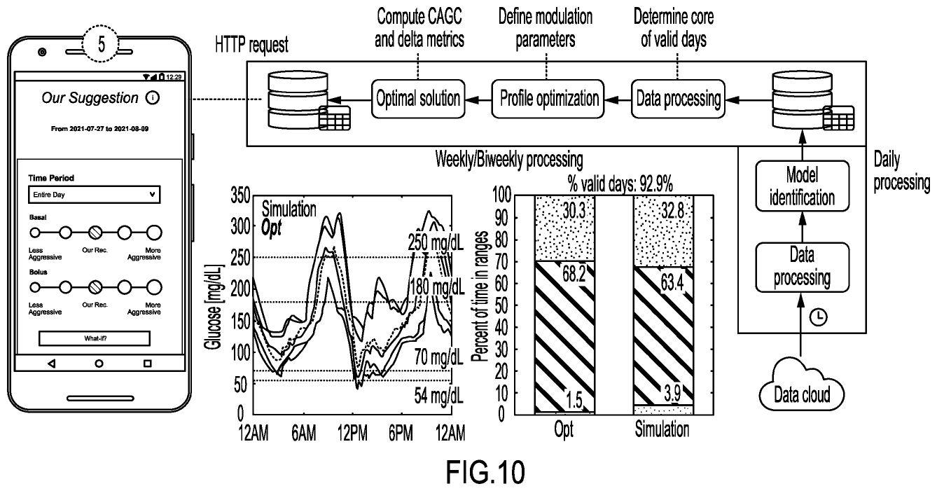

FIG. 10 presents the flow chart of the Web implementation of PAM. The UI screen presents the optimal treatment parameter values calculated by the PAM and their associated glycemic profile and metrics.

Further, the ABC system displays what certain (CAGC) would look like using the BR, CR and CF profiles determined by PAM, and allows the user to then use the Replay Module to simulate hypothetical changes to these profiles by inputting hypothetical treatment changes (FIG. 11). At the top of the screen, users can find the simulator controls: A dropdown to select the entire day or a particular time segment, and two sliders to adjust the aggressiveness of the basal and bolus settings. At the bottom of the screen, users can ask WIT to generate a table listing all the pump settings in the exact same format as they would need to enter into their pumps.

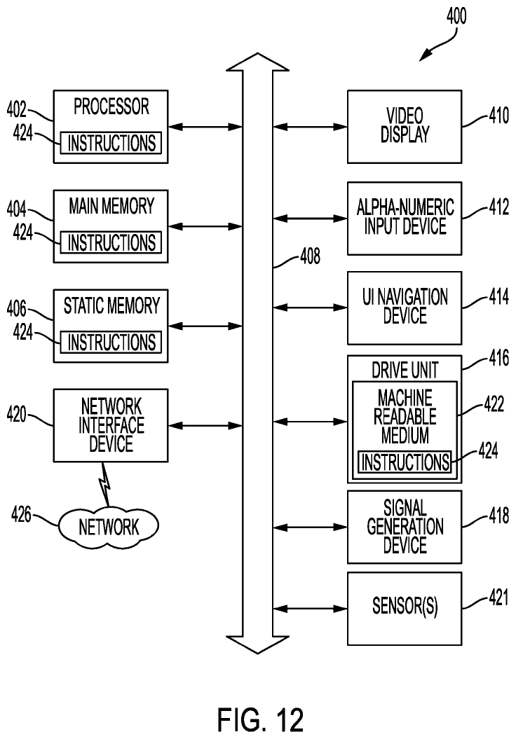

FIG. 12 is a block diagram illustrating an example of a machine upon which one or more aspects of embodiments of the present invention can be implemented.

Referring to FIG. 12, an aspect of an embodiment of the present invention includes, but not limited thereto, a system, method, and computer readable medium for adaptive bio-behavioral control in metabolic disorders that comprises the following: a) PAM which processes a user's historical data to estimate a personalized model of the user's glucose metabolism and to calculate suggested insulin-dosing parameters, b) BAM which assists the user's adaptation to the treatment action by determining a plurality of actionable information and risk assessments based on the user's glucose monitoring records, and c) a Replay Module which computes a simulation of effects from hypothetical changes to the user's treatment or behavior based on a personalized model of the user's glucose metabolism. FIG. 12 illustrates a block diagram of an example machine 400 upon which one or more embodiments (e.g., discussed methodologies) can be implemented (e.g., run).

Examples of machine 400 can include logic, one or more components, circuits (e.g., modules), or mechanisms. Circuits are tangible entities configured to perform certain operations. In an example, circuits can be arranged (e.g., internally or with respect to external entities such as other circuits) in a specified manner. In an example, one or more computer systems (e.g., a standalone, client or server computer system) or one or more hardware processors (processors) can be configured by software (e.g., instructions, an application portion, or an application) as a circuit that operates to perform certain operations as described herein. In an example, the software can reside (1) on a non-transitory machine readable medium or (2) in a transmission signal. In an example, the software, when executed by the underlying hardware of the circuit, causes the circuit to perform the certain operations.

In an example, a circuit can be implemented mechanically or electronically. For example, a circuit can comprise dedicated circuitry or logic that is specifically configured to perform one or more techniques such as discussed above, such as including a special-purpose processor, a field programmable gate array (FPGA) or an application-specific integrated circuit (ASIC). In an example, a circuit can comprise programmable logic (e.g., circuitry, as encompassed within a general-purpose processor or other programmable processor) that can be temporarily configured (e.g., by software) to perform the certain operations. It will be appreciated that the decision to implement a circuit mechanically (e.g., in dedicated and permanently configured circuitry), or in temporarily configured circuitry (e.g., configured by software) can be driven by cost and time considerations.

Accordingly, the term “circuit” is understood to encompass a tangible entity, be that an entity that is physically constructed, permanently configured (e.g., hardwired), or temporarily (e.g., transitorily) configured (e.g., programmed) to operate in a specified manner or to perform specified operations. In an example, given a plurality of temporarily configured circuits, each of the circuits need not be configured or instantiated at any one instance in time. For example, where the circuits comprise a general-purpose processor configured via software, the general-purpose processor can be configured as respective different circuits at different times. Software can accordingly configure a processor, for example, to constitute a particular circuit at one instance of time and to constitute a different circuit at a different instance of time.

In an example, circuits can provide information to, and receive information from, other circuits. In this example, the circuits can be regarded as being communicatively coupled to one or more other circuits. Where multiple of such circuits exist contemporaneously, communications can be achieved through signal transmission (e.g., over appropriate circuits and buses) that connect the circuits. In embodiments in which multiple circuits are configured or instantiated at different times, communications between such circuits can be achieved, for example, through the storage and retrieval of information in memory structures to which the multiple circuits have access. For example, one circuit can perform an operation and store the output of that operation in a memory device to which it is communicatively coupled. A further circuit can then, at a later time, access the memory device to retrieve and process the stored output. In an example, circuits can be configured to initiate or receive communications with input or output devices and can operate on a resource (e.g., a collection of information).

The various operations of method examples described herein can be performed, at least partially, by one or more processors that are temporarily configured (e.g., by software) or permanently configured to perform the relevant operations. Whether temporarily or permanently configured, such processors can constitute processor-implemented circuits that operate to perform one or more operations or functions. In an example, the circuits referred to herein can comprise processor-implemented circuits.

Similarly, the methods described herein can be at least partially processor-implemented. For example, at least some of the operations of a method can be performed by one or processors or processor-implemented circuits. The performance of certain of the operations can be distributed among the one or more processors, not only residing within a single machine, but deployed across a number of machines. In an example, the processor or processors can be located in a single location (e.g., within a home environment, an office environment or as a server farm), while in other examples the processors can be distributed across a number of locations.

The one or more processors can also operate to support performance of the relevant operations in a “cloud computing” environment or as a “software as a service” (SaaS). For example, at least some of the operations can be performed by a group of computers (as examples of machines including processors), with these operations being accessible via a network (e.g., the Internet) and via one or more appropriate interfaces (e.g., Application Program Interfaces (APIs)).

Example embodiments (e.g., apparatus, systems, or methods) can be implemented in digital electronic circuitry, in computer hardware, in firmware, in software, or in any combination thereof. Example embodiments can be implemented using a computer program product (e.g., a computer program, tangibly embodied in an information carrier or in a machine readable medium, for execution by, or to control the operation of, data processing apparatus such as a programmable processor, a computer, or multiple computers).

A computer program can be written in any form of programming language, including compiled or interpreted languages, and it can be deployed in any form, including as a stand-alone program or as a software module, subroutine, or other unit suitable for use in a computing environment. A computer program can be deployed to be executed on one computer or on multiple computers at one site or distributed across multiple sites and interconnected by a communication network.

In an example, operations can be performed by one or more programmable processors executing a computer program to perform functions by operating on input data and generating output. Examples of method operations can also be performed by, and example apparatus can be implemented as, special purpose logic circuitry (e.g., FPGA or ASIC).

The computing system can include clients and servers. A client and server are generally remote from each other and generally interact through a communication network. The relationship of client and server arises by virtue of computer programs running on the respective computers and having a client-server relationship to each other. In embodiments deploying a programmable computing system, it will be appreciated that both hardware and software architectures require consideration. Specifically, it will be appreciated that the choice of whether to implement certain functionality in permanently configured hardware (e.g., an ASIC), in temporarily configured hardware (e.g., a combination of software and a programmable processor), or a combination of permanently and temporarily configured hardware can be a design choice. Below are set out hardware (e.g., machine 400) and software architectures that can be deployed in example embodiments.

In an example, the machine 400 can operate as a standalone device or the machine 400 can be connected (e.g., networked) to other machines.

In a networked deployment, the machine 400 can operate in the capacity of either a server or a client machine in server-client network environments. In an example, machine 400 can act as a peer machine in peer-to-peer (or other distributed) network environments. The machine 400 can be a personal computer (PC), a tablet PC, a set-top box (STB), a Personal Digital Assistant (PDA), a mobile telephone, a web appliance, a network router, switch or bridge, or any machine capable of executing instructions (sequential or otherwise) specifying actions to be taken (e.g., performed) by the machine 400. Further, while only a single machine 400 is illustrated, the term “machine” shall also be taken to include any collection of machines that individually or jointly execute a set (or multiple sets) of instructions to perform any one or more of the methodologies discussed herein.

Example machine (e.g., computer system) 400 can include a processor 402 (e.g., a central processing unit (CPU), a graphics processing unit (GPU) or both), a main memory 404 and a static memory 406, some or all of which can communicate with each other via a bus 408. The machine 400 can further include a display unit 410, an alphanumeric input device 412 (e.g., a keyboard), and a user interface (UI) navigation device 411 (e.g., a mouse). In an example, the display unit 410, input device 412 and UI navigation device 414 can be a touch screen display. The machine 400 can additionally include a storage device (e.g., drive unit) 416, a signal generation device 418 (e.g., a speaker), a network interface device 420, and one or more sensors 421, such as a global positioning system (GPS) sensor, compass, accelerometer, or other sensor.

The storage device 416 can include a machine readable medium 422 on which is stored one or more sets of data structures or instructions 424 (e.g., software) embodying or utilized by any one or more of the methodologies or functions described herein. The instructions 424 can also reside, completely or at least partially, within the main memory 404, within static memory 406, or within the processor 402 during execution thereof by the machine 400. In an example, one or any combination of the processor 402, the main memory 404, the static memory 406, or the storage device 416 can constitute machine readable media.

While the machine readable medium 422 is illustrated as a single medium, the term “machine readable medium” can include a single medium or multiple media (e.g., a centralized or distributed database, and/or associated caches and servers) that configured to store the one or more instructions 424. The term “machine readable medium” can also be taken to include any tangible medium that is capable of storing, encoding, or carrying instructions for execution by the machine and that cause the machine to perform any one or more of the methodologies of the present disclosure or that is capable of storing, encoding or carrying data structures utilized by or associated with such instructions. The term “machine readable medium” can accordingly be taken to include, but not be limited to, solid-state memories, and optical and magnetic media. Specific examples of machine readable media can include non-volatile memory, including, by way of example, semiconductor memory devices (e.g., Electrically Programmable Read-Only Memory (EPROM), Electrically Erasable Programmable Read-Only Memory (EEPROM)) and flash memory devices; magnetic disks such as internal hard disks and removable disks; magneto-optical disks; and CD-ROM and DVD-ROM disks.

The instructions 424 can further be transmitted or received over a communications network 426 using a transmission medium via the network interface device 420 utilizing any one of a number of transfer protocols (e.g., frame relay, IP, TCP, UDP, HTTP, etc.). Example communication networks can include a local area network (LAN), a wide area network (WAN), a packet data network (e.g., the Internet), mobile telephone networks (e.g., cellular networks), Plain Old Telephone (POTS) networks, and wireless data networks (e.g., IEEE 802.11 standards family known as Wi-Fi®, IEEE 802.16 standards family known as WiMax®), peer-to-peer (P2P) networks, among others. The term “transmission medium” shall be taken to include any intangible medium that is capable of storing, encoding or carrying instructions for execution by the machine, and includes digital or analog communications signals or other intangible medium to facilitate communication of such software.

Any of the specified time periods, durations, frequencies, intervals, time of day, and percentages disclosed herein may be varied to meet the operational requirements of the embodiments of the present invention and, of course, be employed within the context of the embodiments of the present invention.

Although example embodiments of the present disclosure are explained in some instances in detail herein, it is to be understood that other embodiments are contemplated. Accordingly, it is not intended that the present disclosure be limited in its scope to the details of construction and arrangement of components set forth in the following description or illustrated in the drawings. The present disclosure is capable of other embodiments and of being practiced or carried out in various ways.

It should be appreciated that any element, part, section, subsection, or component described with reference to any specific embodiment above may be incorporated with, integrated into, or otherwise adapted for use with any other embodiment described herein unless specifically noted otherwise or if it should render the embodiment device non-functional. Likewise, any step described with reference to a particular method or process may be integrated, incorporated, or otherwise combined with other methods or processes described herein unless specifically stated otherwise or if it should render the embodiment method nonfunctional. Furthermore, multiple embodiment devices or embodiment methods may be combined, incorporated, or otherwise integrated into one another to construct or develop further embodiments of the invention described herein.

It should be appreciated that any of the components or modules referred to with regards to any of the present invention embodiments discussed herein, may be integrally or separately formed with one another. Further, redundant functions or structures of the components or modules may be implemented. Moreover, the various components may be communicated locally and/or remotely with any user/clinician/patient or machine/system/computer/processor. Moreover, the various components may be in communication via wireless and/or hardwire or other desirable and available communication means, systems and hardware. Moreover, various components and modules may be substituted with other modules or components that provide similar functions.

It should be appreciated that the device and related components discussed herein may take on all shapes along the entire continual geometric spectrum of manipulation of x, y and z planes to provide and meet the anatomical, environmental, and structural demands and operational requirements. Moreover, locations and alignments of the various components may vary as desired or required.

It should be appreciated that various sizes, dimensions, contours, rigidity, shapes, flexibility and materials of any of the components or portions of components in the various embodiments discussed throughout may be varied and utilized as desired or required.

It should be appreciated that while some dimensions are provided on the aforementioned figures, the device may constitute various sizes, dimensions, contours, rigidity, shapes, flexibility and materials as it pertains to the components or portions of components of the device, and therefore may be varied and utilized as desired or required.

It must also be noted that, as used in the specification and the appended claims, the singular forms “a,” “an” and “the” include plural referents unless the context clearly dictates otherwise. Ranges may be expressed herein as from “about” or “approximately” one particular value and/or to “about” or “approximately” another particular value. When such a range is expressed, other exemplary embodiments include from the one particular value and/or to the other particular value.

By “comprising” or “containing” or “including” is meant that at least the named compound, element, particle, or method step is present in the composition or article or method, but does not exclude the presence of other compounds, materials, particles, or method steps, even if the other such compounds, material, particles, or method steps have the same function as what is named.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the present disclosure. Similarly, it is also to be understood that the mention of one or more components in a device or system does not preclude the presence of additional components or intervening components between those components expressly identified.

Some references, which may include various patents, patent applications, and publications, are cited in a reference list and discussed in the disclosure provided herein. The citation and/or discussion of such references is provided merely to clarify the description of the present disclosure and is not an admission that any such reference is “prior art” to any aspects of the present disclosure described herein. In terms of notation, “[n]” corresponds to the n^{th} reference in the list. All references cited and discussed in this specification are incorporated herein by reference in their entireties and to the same extent as if each reference was individually incorporated by reference.

It should be appreciated that as discussed herein, a subject may be a human or any animal. It should be appreciated that an animal may be a variety of any applicable type, including, but not limited thereto, mammal, veterinarian animal, livestock animal or pet type animal, etc. As an example, the animal may be a laboratory animal specifically selected to have certain characteristics similar to human (e.g. rat, dog, pig, monkey), etc. It should be appreciated that the subject may be any applicable human patient, for example.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 10% of the numerical value of the number with which it is being used. Therefore, about 50% means in the range of 45%-55%. Numerical ranges recited herein by endpoints include all numbers and fractions subsumed within that range (e.g. 1 to 5 includes 1, 1.5, 2, 2.75, 3, 3.90, 4, 4.24, and 5). Similarly, numerical ranges recited herein by endpoints include subranges subsumed within that range (e.g. 1 to 5 includes 1-1.5, 1.5-2, 2-2.75, 2.75-3, 3-3.90, 3.90-4, 4-4.24, 4.24-5, 2-5, 3-5, 1-4, and 2-4). It is also to be understood that all numbers and fractions thereof are presumed to be modified by the term “about.”

### EXAMPLES

Practice of an aspect of an embodiment (or embodiments) of the invention will be still more fully understood from the following examples and experimental results, which are presented herein for illustration only and should not be construed as limiting the invention in any way.

Example 1. A processor-based method for improving human-machine co-regulation of metabolic disorders, the method comprising:
receiving a user's historical data, the user's historical data including the user's glucose monitoring records and insulin therapy profile;
determining a plurality of actionable information and risk assessments to assist the user's behavioral intervention based on user's glucose monitoring records;
processing said historical data to estimate a personalized model of the user's glucose metabolism;
determining insulin-dosing parameters based on said personalized model of the user's glucose metabolism; and
computing a simulation of effects from hypothetical changes to the user's treatment or behavior based on said personalized model of the user's glucose metabolism.

Example 2. The method of example 1, wherein the plurality of actionable information and risk assessments include one or more of the following:
the user's time spent in different glucose ranges;
a glucose variability index which tracks daily glucose variation;
the user's glucose median and percentiles across a period of time;
the user's time spent in different glucose ranges;
an estimate mean glucose (EMG); and
the user's hypoglycemia and hyperglycemia risk indices.

Example 3. The method of example 2, wherein the time spent in different glucose ranges, glucose variability index, and glucose median and percentiles are computed only when there is at least 70% of continuous glucose monitoring records for the time period of interest.

Example 4. The method of example 2 (as well as subject matter in whole or in part of example 3), wherein, every 24 hours (or other specified duration), the estimate mean glucose is computed from received historical data, wherein said historical data includes at least 70% of continuous glucose monitoring from a closed time period prior to the calculation. In an embodiment, the specified durations may be any variety as desired or required, such as, but not limited thereto, 4 hours, 8 hours, 12 hours, 24, hours, weekly, etc., or of any combination thereof. The specified durations do not need to be equally spaced in time.

Example 5. The method of example 4 (as well as subject matter of one or more of any combination of examples 2-3, in whole or in part), wherein the formula used to compute the estimate mean glucose is:

$$EMG ( \% ) = 3.31 + 0.02392 \times [ mean glucose in mg / dl ] .$$

Example 6. The method of example 2 (as well as subject matter of one or more of any combination of examples 3-5, in whole or in part), wherein, if less than 70% of continuous glucose monitoring data from the preceding 14 days (or other specified preceding duration) is available, the trend of the estimate mean glucose change over the previous seven days is estimated through linear regression and mapped into a specific “arrow slope,” wherein said arrow slope represents the estimate mean glucose rate of change. In an embodiment, the specified preceding durations may be any variety as desired or required, such as, but not limited thereto, weekly, every 10 days, or monthly, etc., or of any combination thereof. The specified preceding durations may be implemented on demand by the user.

Example 7. The method of example 6 (as well as subject matter of one or more of any combination of examples 2-5, in whole or in part), wherein the estimate mean glucose rate of change (emgROC) is based on the lookup table:

| emgROC | Arrow Slope |
|---|---|
| <=a, | Rapid decrease |
| where a < −1.0 mg/dL/min | (code: −2) |
| >a AND <=b, | Moderate decrease |
| where a < −1.0 mg/dL/min | (code: −1) |
| where b is between −1.0 and −0.25 mg/dL/min | |
| >b AND <=c, | No relevant change |
| where b is between −1.0 and −0,25 mg/dL/min | (code: 0) |
| where c is between 0.25 and 1.0 mg/dL/min | |
| >c AND <=d, | Moderate increase |
| where c is between 0.25 and 1.0 mg/dL/min | (code: +1) |
| where d > 1 mg/dL/min | |
| >=d, | Rapid increase |
| where d > 1.0 mg/dL/min. | (code: +2) |

Example 8. The method of example 2 (as well as subject matter of one or more of any combination of examples 3-7, in whole or in part), wherein the formulas used to compute said hypoglycemia risk index (low blood glucose index (LBGI)) and said hyperglycemia risk index (high blood glucose index (HBGI)) are:

$$LBGI = \frac{\sum {r}_{l} ( {x}_{i} )}{n}$$

where rₗ(xᵢ)=22.77f(xᵢ)² if f(xᵢ)≤0, and 0 otherwise; and

$$HBGI = \frac{\sum {r}_{h} ( {x}_{i} )}{n}$$

where rₕ(x)=22.77f(xᵢ)² if f(xᵢ)>0, and 0 otherwise, and where
f(xᵢ)=(ln(xᵢ)^{1.804}-5,381 3for blood glucose readings x₁ . . . , xₙ measured in mg/dL.

Example 9. The method of example 1 (as well as subject matter of one or more of any combination of examples 2-8, in whole or in part), wherein said historical data further includes the user's meal history and/or controller-specific records.

Example 10. The method of example 1 (as well as subject matter of one or more of any combination of examples 2-9, in whole or in part), wherein said historical data is parsed into “valid extended days” with each extended day being obtained from a 24-hour calendar day by adding a 6-hour head and a 2-hour tail. In an embodiment, the specified head and tail duration may be any variety as desired or required.

Example 11. The method of example 1 (as well as subject matter of one or more of any combination of examples 2-10, in whole or in part), further comprising: executing a logistic regression-based detection algorithm to reconstruct and amend a meal record of the user.

Example 12. The method of example 10 (as well as subject matter in whole or in part of example 11), wherein said extended days are marked “valid” if:
continuous glucose monitoring gaps are no longer than 3 hours;
at least 70% of continuous glucose monitoring data are available; and
at least 2 boluses are recorded. In an embodiment, the monitoring gaps may be any variety as desired or required, such as, but not limited thereto, 30 minutes, 1 hour, 2 hours, etc., or of any combination thereof. In an embodiment, the boluses may be any variety as desired or required, such as, but not limited thereto, 0, 1, or 3, etc.

Example 13. The method of example 10 (as well as subject matter of one or more of any combination of examples 2-9 and 11-12, in whole or in part), further comprising: identifying, for each “valid extended day”, said personalized model of the user's glucose metabolism in the presence of meal ingestion and subcutaneous insulin administration.

Example 14. The method of example 1 (as well as subject matter of one or more of any combination of examples 2-13, in whole or in part), wherein said personalized model is identified daily. In an embodiment, the identification may be executed as desired or required, such as, but not limited thereto, daily, every 2-3 days, weekly, etc. In an embodiment, the identification may be executed on demand by the user as desired or required.

Example 15. The method of example 1 (as well as subject matter of one or more of any combination of examples 2-14, in whole or in part), further comprising: estimating an additive net-effect signal through model inversion by deconvolution based on said personalized model.

Example 16. The method of example 11 (as well as subject matter of one or more of any combination of examples 2-10 and 12-15, in whole or in part), wherein the personalized model of the user's glucose metabolism comprises:

identifying a personalized model of the user's glucose metabolism in the presence of meal ingestion and subcutaneous insulin administration; and

estimating an additive net-effect signal through model inversion by deconvolution.

Example 17. The method of example 15 (as well as subject matter in whole or in part of example 16), further comprising: recording processed data, model parameters, and net-effect signal in a system database.

Example 18. The method of example 13 (as well as subject matter of one or more of any combination of examples 2-12 and 14-17, in whole or in part), further comprising: periodically replaying glucose traces represented by the personalized model of the user's glucose metabolism in a closed-loop fashion with an automated insulin delivery system.

Example 19. The method of example 18 (as well as subject matter of one or more of any combination of examples 2-17, in whole or in part), further comprising: associating said replayed glucose traces with a root mean square error.

Example 20. The method of example 19 (as well as subject matter of one or more of any combination of examples 2-18, in whole or in part), wherein said glucose traces with a root mean square error above a predefined threshold are excluded from the dataset used to determine insulin-dosing parameters.

Example 21. The method of example 20 (as well as subject matter of one or more of any combination of examples 2-19, in whole or in part), wherein said predefined threshold is 25 mg/dL. In an embodiment, the predefined threshold may be any variety as desired or required. Example 22. The method of example 1 (as well as subject matter of one or more of any combination of examples 2-21, in whole or in part), wherein said insulin-dosing parameters are determined by solving an optimization problem that minimizes the weighted sum of the risks of hypoglycemia (LBGI) and hyperglycemia (HBGI) and the number of hypoglycemic treatments.

Example 23. The method of example 1 (as well as subject matter of one or more of any combination of examples 2-22, in whole or in part), wherein said insulin-dosing parameters are determined by solving the optimization problem:

$${min}_{\{ {α}_{1} , … , {α}_{6} \} \{ {β}_{1} , … , {β}_{6} \}} J = f ( {BR}^{′} , {CR}^{′} , {CF}^{′} )$$

$$subject to$$

$${α}_{i} ∈ [ - 1 , 1 ] ∀ i = 1 , … , 6$$

$${β}_{i} ∈ [ - 1 , 1 ] ∀ i = 1 , … , 6$$

with α^{v}={α₁, . . . , α₆} and β^{v}+={β₁, . . . β₆} the vector of modulation factors for CR-CF and BR in segments with predefined lengths, and BR′, CR′, and CF′, the modulated profiles through the day for BR and CR-CF computed as a function of α^{v} and β^{v}:

$${BR}^{′} = {BR}_{nom}^{v} \cdot ( 1 + P * \frac{{{β}_{ext}}^{v}}{100} )$$

$${CR}^{′} = {CR}_{nom}^{v} \cdot ( 1 + P * \frac{{α}_{ext}^{v}}{100} )$$

$${CF}^{′} = {CF}_{nom}^{v} \cdot ( 1 + P * \frac{{α}_{ext}^{v}}{100} )$$

with

$${BR}_{nom}^{v} , {CR}_{nom}^{v} and {CF}_{nom}^{v}$$

the current profiles through the day for BR, CR, and CF in five-minute intervals, and

$${α}_{ext}^{v}$$

and βₑₓₜ^{v} the extended version of α^{v} and β^{v} through the day in five-minute intervals; and
where the cost function (J) is:

$$J = {\sum }_{j = 1}^{{N}_{days}} 2 \cdot {LBGI}_{j} + {HBGI}_{j} + {N}_{j , HT}$$

with N_{days} the number of days considered for the optimization (≤14 days), N_{HT} the number of hypoglycemic treatments per day, and LBGIⱼ and HBGIⱼ the hypoglycemia and hyperglycemia risk indices per day, computed as:

$${Risk}_{k} = log {BG}_{k}^{1.084} - 5.381$$

$${LBGI}_{j} = {\sum }_{k = 1}^{{N}_{samples}} 22.77 * {Risk}_{k}^{2} ∀ {Risk}_{k} \leq 0 and {BG}_{k} < 70$$

$${HBGI}_{j} = {\sum }_{k = 1}^{{N}_{samples}} 22.77 * {Risk}_{k}^{2} ∀ {Risk}_{k} > 0 and {BG}_{k} < 180$$

with Nₛₐₘₚₗₑₛ=288 the number of samples per day considering a sampling time equal to five minutes.

Example 24. The method of example 1 (as well as subject matter of one or more of any combination of examples 2-23, in whole or in part), wherein the historical data further includes a finite time range and basal and bolus modulators.

Example 25. The method of example 1 (as well as subject matter of one or more of any combination of examples 2-24, in whole or in part), wherein the said simulation is based on custom modulation factors.

Example 26. The method of example 25, wherein said custom modulation factors are determined by parsing received historical data into finite time ranges with associated basal and bolus modulators based on said personalized model of the user's glucose metabolism. Example 27. The method of example 25 (as well as subject matter in whole or in part of example 26), wherein said custom modulation factors are defined as Pfactors.

Example 28. The method of example 27 (as well as subject matter of one or more of any combination of examples 2-26, in whole or in part), wherein the said simulation is based on combining optimal modulation factors with said Pfactors to define final modulation profiles for at least one of: basal rate, carbohydrate ratio, and correction factor.

Example 29. The method of example 1 (as well as subject matter of one or more of any combination of examples 2-28, in whole or in part), wherein said simulation features changes to at least one of: basal rate profile, carbohydrate ratio profile, correction factor profiles, or user-specific glucose charts.

Example 30. The method of example 1 (as well as subject matter of one or more of any combination of examples 2-29, in whole or in part), wherein said treatment includes changes to basal and bolus settings.

Example 31. The method of example 1 (as well as subject matter of one or more of any combination of examples 2-30, in whole or in part), further comprising presenting the time spent in different glucose ranges via commonly accepted glucose charts.

Example 32. The method of example 1 (as well as subject matter of one or more of any combination of examples 2-31, in whole or in part), further comprising one or more of the following:
recording said plurality of actionable information and risk assessments to a database;
recording said plurality of actionable information and risk assessments to a database accessible by the user;
presenting said plurality of actionable information and risk assessments to the user via a display system;
presenting said plurality of actionable information and risk assessments to the user via a website or web application;
recording said insulin-dosing parameters based on the personalized model of the user's glucose metabolism to a database;
recording said insulin-dosing parameters based on the personalized model of the user's glucose metabolism to a database accessible by the user,
presenting said insulin-dosing parameters based on the personalized model of the user's glucose metabolism via a display system; and
presenting said insulin-dosing parameters based on the personalized model of the user's glucose metabolism via a website or web application.

Example 33. The method of example 1 (as well as subject matter of one or more of any combination of examples 2-32, in whole or in part), wherein at least part of the historical data is obtained from one or more of the following:
an insulin pump device, continuous glucose monitoring device, a manual insulin injection device, a meal-tracking device, and an activity-tracking device.

Example 34. The method of example I (as well as subject matter of one or more of any combination of examples 2-33, in whole or in part), wherein the insulin-dosing parameters are recommended to the user or automatically implemented by a closed-loop control or continuous subcutaneous insulin delivery system.

Example 35. A system for improving human-machine co-regulation of metabolic disorders, the system comprising:
a computer processor;
a memory configured to store instructions that are executable by said computer processor, wherein said computer processor is configured to execute instructions for:
   receiving a user's historical data, the user's historical data including the user's glucose monitoring records and insulin therapy profile;
   determining a plurality of actionable information and risk assessments to assist the user's behavioral intervention based on user's glucose monitoring records;
   processing said historical data to estimate a personalized model of the user's glucose metabolism;
   determining insulin-dosing parameters based on said personalized model of the user's glucose metabolism; and
   computing a simulation of effects from hypothetical changes or non-changes to the user's treatment or behavior based on said personalized model of the user's glucose metabolism.

Example 36. The system of example 35, wherein the plurality of actionable information and risk assessments include one or more of the following:
the user's time spent in different glucose ranges;
a glucose variability index which tracks daily glucose variation;
the user's glucose median and percentiles across a period of time;
the user's time spent in different glucose ranges;
an estimate mean glucose (EMG); and
the user's hypoglycemia and hyperglycemia risk indices.

Example 37. The system of example 36, wherein the time spent in different glucose ranges, glucose variability index, and glucose median and percentiles are computed only when there is at least 70% of continuous glucose monitoring records for the time period of interest.

Example 38. The system of example 36 (as well as subject matter in whole or in part of example 37), wherein, every 24 hours (or other specified duration), the estimate mean glucose is computed from received historical data, wherein said historical data includes at least 70% of continuous glucose monitoring from a closed time period prior to the calculation. In an embodiment, the specified durations may be any variety as desired or required, such as, but not limited thereto, 4 hours, 8 hours, 12 hours, 24, hours, weekly, etc., or of any combination thereof. The specified durations do not need to be equally spaced in time.

Example 39. The system of example 38 (as well as subject matter of one or more of any combination of examples 36-37, in whole or in part), wherein the formula used to compute the estimate mean glucose is:

$$EMG ( \% ) = 3.31 + 0.02392 \times [ mean glucose in mg / dl ] .$$

Example 40. The system of example 36 (as well as subject matter of one or more of any combination of examples 37-39, in whole or in part), wherein, if less than 70% of continuous glucose monitoring data from the preceding 14 days is available, the trend of the estimate mean glucose change over the previous seven days is estimated through linear regression and mapped into a specific “arrow slope,” wherein said arrow slope represents the estimate mean glucose rate of change. In an embodiment, the specified preceding durations may be any variety as desired or required, such as, but not limited thereto, weekly, every 10 days, or monthly, etc., or of any combination thereof. The specified preceding durations may be implemented on demand by the user.

Example 41. The system of example 40 (as well as subject matter of one or more of any combination of examples 36-39, in whole or in part), wherein the estimate mean glucose rate of change (emgROC) is based on the lookup table:

| emgROC | Arrow Slope |
|---|---|
| <=a, | Rapid decrease |
| where a < −1.0 mg/dL/min | (code: −2) |
| >a AND <=b, | Moderate decrease |
| where a < −1.0 mg/dL/min | (code: −1) |
| where b is between −1.0 and −0.25 mg/dL/min | |
| >b AND <=c, | No relevant change |
| where b is between −1.0 and −0.25 mg/dL/min | (code: 0) |
| where c is between 0.25 and 1.0 mg/dL/min | |
| >c AND <=d, | Moderate increase |
| where c is between 0.25 and 1.0 mg/dL/min | (code: +1) |
| where d > 1 mg/dL/min | |
| >=d, | Rapid increase |
| where d > 1.0 mg/dL/min. | (code: +2) |

Example 42. The system of example 36 (as well as subject matter of one or more of any combination of examples 37-41, in whole or in part), wherein the formulas used to compute said hypoglycemia risk index (low blood glucose index (LBGI)) and said hyperglycemia risk index (high blood glucose index (HBGI)) are:

$$LBGI = \frac{\sum {r}_{l} ( {x}_{i} )}{n}$$

where rₗ(xᵢ)=22.77f(xᵢ)² if f(xᵢ)≤0, and 0 otherwise; and

$$HBGI = \frac{\sum {r}_{h} ( {x}_{i} )}{n}$$

where rₕ(xᵢ)=22.77f(xᵢ)² if f(xᵢ)>0, and 0 otherwise, and where f(xᵢ)=(ln(xᵢ)^{1.804}-5.381 3for blood glucose readings x₁, . . . , xₙ measured in mg/dL.

Example 43. The system of example 35 (as well as subject matter of one or more of any combination of examples 36-42, in whole or in part), wherein said historical data further includes the user's meal history and/or controller-specific records.

Example 44. The system of example 35 (as well as subject matter of one or more of any combination of examples 36-43, in whole or in part), wherein said historical data is parsed into “valid extended days” with each extended day being obtained from a 24-hour calendar day by adding a 6-hour head and a 2-hour tail. In an embodiment, the specified head and tail duration may be any variety as desired or required.

Example 45. The system of example 35 (as well as subject matter of one or more of any combination of examples 36-44, in whole or in part), wherein said computer processor is further configured to execute instructions for:
computing a logistic regression-based detection algorithm to reconstruct and amend a meal record of the user.

Example 46. The system of example 44 (as well as subject matter of one or more of any combination of examples 36-43 and 45, in whole or in part), wherein said extended days are marked “valid” if:
continuous glucose monitoring gaps are no longer than 3 hours;
at least 70% of continuous glucose monitoring data are available; and
at least 2 boluses are recorded. In an embodiment, the monitoring gaps may be any variety as desired or required, such as, but not limited thereto, 30 minutes, 1 hour, 2 hours, etc., or of any combination thereof. In an embodiment, the boluses may be any variety as desired or required, such as, but not limited thereto, 0, 1, or 3, etc.

Example 47. The system of example 44 (as well as subject matter of one or more of any combination of examples 36-43 and 45-46, in whole or in part), wherein said computer processor is further configured to execute instructions for:
identifying, for each valid extended day, said personalized model of the user's glucose metabolism in the presence of meal ingestion and subcutaneous insulin administration.

Example 48. The system of example 35 (as well as subject matter of one or more of any combination of examples 36-47, in whole or in part), wherein said personalized model is identified daily. In an embodiment, the identification may be executed as desired or required, such as, but not limited thereto, daily, every 2-3 days, weekly, etc. In an embodiment, the identification may be executed on demand by the user as desired or required

Example 49. The system of example 35 (as well as subject matter of one or more of any combination of examples 36-48, in whole or in part), wherein said computer processor is further configured to execute instructions for: estimating an additive net-effect signal through model inversion by deconvolution based on said personalized model.

Example 50. The system of example 45 (as well as subject matter of one or more of any combination of examples 36-44 and 46-49, in whole or in part), wherein the personalized model of the user's glucose metabolism comprises:
identifying a personalized model of the user's glucose metabolism in the presence of meal ingestion and subcutaneous insulin administration; and
estimating an additive net-effect signal through model inversion by deconvolution.

Example 51. The system of example 49 (as well as subject matter of one or more of any combination of examples 36-48 and 50, in whole or in part), wherein said computer processor is further configured to execute instructions for: recording processed data, model parameters, and net-effect signal in a system database.

Example 52. The system of example 47 (as well as subject matter of one or more of any combination of examples 36-46 and 48-51, in whole or in part), further configured to execute instructions for: periodically replaying glucose traces represented by the personalized model of the user's glucose metabolism in a closed-loop fashion with an automated insulin delivery system.

Example 53. The system of example 52 (as well as subject matter of one or more of any combination of examples 36-51, in whole or in part), wherein said computer processor is further configured to execute instructions for: associating said replayed glucose traces with a root mean square error.

Example 54. The system of example 53 (as well as subject matter of one or more of any combination of examples 36-52, in whole or in part), wherein said glucose traces with a root mean square error above a predefined threshold are excluded from the dataset used to determine insulin-dosing parameters.

Example 55. The system of example 54 (as well as subject matter of one or more of any combination of examples 36-53, in whole or in part), wherein said predefined threshold is 25 mg/dl In an embodiment, the predefined threshold may be any variety as desired or required.

Example 56. The system of example 35 (as well as subject matter of one or more of any combination of examples 36-55, in whole or in part), wherein said insulin-dosing parameters are determined by solving an optimization problem that minimizes the weighted sum of the risks of hypoglycemia (LBGI) and hyperglycemia (HBGI) and the number of hypoglycemic treatments.

Example 57. The system of example 35 (as well as subject matter of one or more of any combination of examples 36-56, in whole or in part), wherein said insulin-dosing parameters are determined by solving the optimization problem:

$${min}_{\{ {α}_{1} , … , {α}_{6} \} \{ {β}_{1} , … , {β}_{6} \}} J = f ( {BR}^{′} , {CR}^{′} , {CF}^{′} )$$

$$subject to$$

$${α}_{i} ∈ [ - 1 , 1 ] ∀ i = 1 , … , 6$$

$${β}_{i} ∈ [ - 1 , 1 ] ∀ i = 1 , … , 6$$

with α^{v}={α₁, . . . , α₆} and β^{v}={β₁, . . . , β₆} the vector of modulation factors for CR-CF and BR in segments with predefined lengths, and BR′, CR′, and CF′, the modulated profiles through the day for BR and CR-CF computed as a function of α^{v} and β²:

$${BR}^{′} = {BR}_{nom}^{v} \cdot ( 1 + P * \frac{{β}_{ext}^{v}}{100} )$$

$${CR}^{′} = {CR}_{nom}^{v} \cdot ( 1 + P * \frac{{α}_{ext}^{v}}{100} )$$

$${CF}^{′} = {CF}_{nom}^{v} \cdot ( 1 + P * \frac{{α}_{ext}^{v}}{100} )$$

with

$${BR}_{nom}^{v} , {CR}_{nom}^{v} and {CF}_{nom}^{v}$$

the current profiles through the day for BR, CR, and CF in five-minute intervals, and

$${α}_{ext}^{v}$$

and βₑₓₜ^{v} the extended version of α^{v} and β^{v} through the day in five-minute intervals; and
where the cost function (J) is:

$$J = {\sum }_{j = 1}^{{N}_{days}} 2 \cdot {LBGI}_{j} + {HBGI}_{j} + {N}_{j , HT}$$

with N_{days} the number of days considered for the optimization (≤14 days), N_{HT} the number of hypoglycemic treatments per day, and LBGIⱼ and HBGIⱼ the hypoglycemia and hyperglycemia indices per day, computed as:

$${Risk}_{k} = log {BG}_{k}^{1.084} - 5.381$$

$${LBGI}_{j} = {\sum }_{k = 1}^{{N}_{samples}} 22.77 * {Risk}_{k}^{2} ∀ {Risk}_{k} \leq 0 and {BG}_{k} < 70$$

$${HBGI}_{j} = {\sum }_{k = 1}^{{N}_{samples}} 22.77 * {Risk}_{k}^{2} ∀ {Risk}_{k} > 0 and {BG}_{k} > 180$$

with Nₛₐₘₚₗₑₛ=288 the number of samples per day considering a sampling time equal to five minutes. In an embodiment, the number of days considered for the optimization may be any variety as desired or required, such as greater than 14 days.

Example 58. The system of example 35 (as well as subject matter of one or more of any combination of examples 36-57, in whole or in part), wherein the historical data further includes a finite time range and basal and bolus modulators.

Example 59. The system of example 35 (as well as subject matter of one or more of any combination of examples 36-58, in whole or in part), wherein the said simulation is based on custom modulation factors.

Example 60. The system of example 59 (as well as subject matter of one or more of any combination of examples 36-58, in whole or in part), wherein said custom modulation factors are determined by parsing received historical data into finite time ranges with associated basal and bolus modulators based on said personalized model of the user's glucose metabolism.

Example 61. The system of example 59 (as well as subject matter in whole or in part of example 60), wherein said custom modulation factors are defined as Pfactors.

Example 62. The system of example 61 (as well as subject matter of one or more of any combination of examples 36-60, in whole or in part), wherein the said simulation is based on combining optimal modulation factors with said Pfactors to define final modulation profiles for at least one of: basal rate, carbohydrate ratio, and correction factor.

Example 63. The system of example 35 (as well as subject matter of one or more of any combination of examples 36-62, in whole or in part), wherein said simulation features changes or non-changes to at least one of: basal rate profile, carbohydrate ratio profile, correction factor profiles, or user-specific glucose charts.

Example 64. The system of example 35 (as well as subject matter of one or more of any combination of examples 36-63, in whole or in part), wherein said treatment includes changes or non-changes to basal and bolus settings.

Example 65. The system of example 35 (as well as subject matter of one or more of any combination of examples 36-64, in whole or in part), further comprising presenting the time spent in different glucose ranges via commonly accepted glucose charts.

Example 66. The system of example 35 (as well as subject matter of one or more of any combination of examples 36-65, in whole or in part), wherein said computer processor is further configured to execute instructions for one or more of the following:
recording said plurality of actionable information and risk assessments to a database;
recording said plurality of actionable information and risk assessments to a database accessible by the user;
presenting said plurality of actionable information and risk assessments to the patient via a display system;
presenting said plurality of actionable information and risk assessments to the user via a website or web application;
recording said insulin-dosing parameters based on the personalized model of the user's glucose metabolism to a database;
recording said insulin-dosing parameters based on the personalized model of the user's glucose metabolism to a database accessible by the user;
presenting said insulin-dosing parameters based on the personalized model of the user's glucose metabolism via a display system; and
presenting said insulin-dosing parameters based on the personalized model of the user's glucose metabolism via a website or web application.

Example 67. The system of example 35 (as well as subject matter of one or more of any combination of examples 36-66, in whole or in part), wherein said system further comprises one or more of the following:
a portal configured to manage user accounts;
a database used to store user data and programmed instructions for system operation; and
a user interface capable of displaying content and sending notifications to the user.

Example 68. The system of example 67 (as well as subject matter of one or more of any combination of examples 36-66, in whole or in part), wherein said portal is a web-based portal.

Example 69. The system of example 67 (as well as subject matter in whole or in part of example 68), wherein:
said system is configured to communicate with a third-party system.

Example 70. The system of example 69 (as well as subject matter of one or more of any combination of examples 36-68, in whole or in part), wherein:
said third party system is configured to communicate with a server coupled with a network; and
wherein said third-party system is configured to upload the user's historical data to the processor by way of the server.

Example 71. The system of example 69 (as well as subject matter in whole or in part of example 70), wherein said third-party system is connected to the Internet that is capable of connecting to and uploading data from at least one of: an insulin monitoring device, an insulin delivery device, or a glucose monitoring device.

Example 72. The system of example 69 (as well as subject matter of one or more of any combination of examples 36-68 and 70-71, in whole or in part), wherein said third party system includes one or more of the following: mobile app, tablet, computer, mobile device, personal electronic device, personal digital assistant (PDA), or smartwatch.

Example 73 The system of example 35 (as well as subject matter of one or more of any combination of examples 36-72, in whole or in part), wherein at least part of the historical data is obtained or uploaded from one or more of the following:
an insulin pump device, continuous glucose monitoring device, a manual insulin injection device, a meal-tracking device, and an activity-tracking device.

Example 74. The system of example 35 (as well as subject matter of one or more of any combination of examples 36-73, in whole or in part), wherein the insulin-dosing parameters are recommended to the user or automatically implemented by a closed-loop control or continuous subcutaneous insulin delivery system.

Example 75. A non-transitory computer readable medium having program instructions for use in human-machine co-regulation of metabolic disorders, wherein execution of the program instructions by one or more processors of a computer system causes the one or more processors to:
receive a user's historical data, the user's historical data including the user's glucose monitoring records and insulin therapy profile;
determine a plurality of actionable information and risk assessments to assist the user's behavioral intervention based on user's glucose monitoring records;
process said historical data to estimate a personalized model of the user's glucose metabolism;
determine insulin-dosing parameters based on said personalized model of the user's glucose metabolism; and
compute a simulation of effects from hypothetical changes to the user's treatment or behavior based on said personalized model of the user's glucose metabolism.

Example 76. The non-transitory computer readable medium of example 75, wherein execution of the program instructions by one or more processors of the computer system further causes the one or more processors to:
communicate with a third party system.

Example 77. The non-transitory computer readable medium of example 76, wherein;
said third party system is configured to communicate with a server coupled to a network, and
said third-party system is configured to upload the user's historical data to the one or more processors by way of the server.

Example 78. The non-transitory computer readable medium of example 76 (as well as subject matter in whole or in part of example 77), wherein execution of the program instructions by one or more processors of the computer system further causes the one or more processors to: display content and send notifications to the user via a user interface.

Example 79. The non-transitory computer readable medium of example 76 (as well as subject matter of one or more of any combination of examples 77-78, in whole or in part), wherein said third-party system connected to the Internet that is capable of connecting to and uploading data from at least one of:
an insulin monitoring device, an insulin delivery device, or a glucose monitoring device.

Example 80. The non-transitory computer readable medium of example 76 (as well as subject matter of one or more of any combination of examples 77-79, in whole or in part), wherein said third party system includes one or more of the following: mobile app, tablet, computer, mobile device, personal electronic device, personal digital assistant (PDA), or smartwatch.

Example 81. The non-transitory computer readable medium of example 75 (as well as subject matter of one or more of any combination of examples 76-80, in whole or in part), wherein the plurality of actionable information and risk assessments include one or more of the following:
the user's time spent in different glucose ranges;
a glucose variability index which tracks daily glucose variation;
the user's glucose median and percentiles across a period of time;
the user's time spent in different glucose ranges;
an estimate mean glucose (EMG); and
the user's hypoglycemia and hyperglycemia risk indices.

Example 82. The non-transitory computer readable medium of example 81 (as well as subject matter of one or more of any combination of examples 76-79, in whole or in part), wherein the time spent in different glucose ranges, glucose variability index, and glucose median and percentiles are computed only when there is at least 70% of continuous glucose monitoring records for the time period of interest.

Example 83. The system of example 81 (as well as subject matter in whole or in part of example 82), wherein, every 24 hours (or other specified duration), the estimate mean glucose is computed from received historical data, wherein said historical data includes at least 70% of continuous glucose monitoring from a closed time period prior to the calculation. In an embodiment, the specified durations may be any variety as desired or required, such as, but not limited thereto, 4 hours, 8 hours, 12 hours, 24, hours, weekly, etc., or of any combination thereof. The specified durations do not need to be equally spaced in time.

Example 84. The non-transitory computer readable medium of example 83 (as well as subject matter of one or more of any combination of examples 76-82, in whole or in part), wherein the formula used to compute the estimate mean glucose is:

$$EMG ( \% ) = 3.31 + 0.02392 \times [ mean glucose in mg / dl ] .$$

Example 85. The non-transitory computer readable medium of example 81 (as well as subject matter of one or more of any combination of examples 76-80 and 82-84, in whole or in part), wherein, if less than 70% of continuous glucose monitoring data from the preceding 14 days is available, the trend of the estimate mean glucose change over the previous seven days is estimated through linear regression and mapped into a specific “arrow slope,” wherein said arrow slope represents the estimate mean glucose rate of change. In an embodiment, the specified preceding durations may be any variety as desired or required, such as, but not limited thereto, weekly, every 10 days, or monthly, etc., or of any combination thereof. The specified preceding durations may be implemented on demand by the user.

Example 86. The non-transitory computer readable medium of example 85 (as well as subject matter of one or more of any combination of examples 76-84, in whole or in part), wherein the estimate mean glucose rate of change (emgROC) is based on the lookup table:

| emgROC | Arrow Slope |
|---|---|
| <=a, | Rapid decrease |
| where a < −1.0 mg/dL/min | (code: −2) |
| >a AND <=b, | Moderate decrease |
| where a < −1.0 mg/dL/min | (code: −1) |
| where b is between −1.0 and −0.25 mg/dL/min | |
| >b AND <=c, | No relevant change |
| where b is between −1.0 and −0,25 mg/dL/min | (code: 0) |
| where c is between 0.25 and 1.0 mg/dL/min | |
| >c AND <=d, | Moderate increase |
| where c is between 0.25 and 1.0 mg/dL/min | (code: +1) |
| where d > 1 mg/dL/min | |
| >=d, | Rapid increase |
| where d > 1.0 mg/dL/min. | (code: +2) |

Example 87. The non-transitory computer readable medium of example 81 (as well as subject matter of one or more of any combination of examples 76-80 and 82-86, in whole or in part), wherein the formulas used to compute said hypoglycemia risk index (low blood glucose index (LBGI)) and said hyperglycemia risk index (high blood glucose index (HBGI)) are:

$$LBGI = \frac{\sum {r}_{l} ( {x}_{i} )}{n}$$

where rₗ(xₗ)=22.77f(xᵢ)² if f(xᵢ)≤0, and 0 otherwise; and

$$HBGI = \frac{\sum {r}_{h} ( {x}_{i} )}{n}$$

where rₕ(xᵢ)=22.77f(xᵢ)² if f(xᵢ)>0, and 0 otherwise, and where f(xᵢ)=(ln(xᵢ)^{1.804}-5.381 3for blood glucose readings x₁, . . . , xₙ measured in mg/dL.

Example 88. The non-transitory computer readable medium of example 75 (as well as subject matter of one or more of any combination of examples 76-87, in whole or in part), wherein said historical data further includes the user's meal history and/or controller-specific records.

Example 89. The non-transitory computer readable medium of example 75 (as well as subject matter of one or more of any combination of examples 76-88, in whole or in part), wherein said historical data is parsed into “valid extended days” with each extended day being obtained from a 24-hour calendar day by adding a 6-hour head and a 2-hour tail. In an embodiment, the specified head and tail duration may be any variety as desired or required.

Example 90. The non-transitory computer readable medium of example 75 (as well as subject matter of one or more of any combination of examples 76-89, in whole or in part), wherein said computer processor is further configured to execute instructions for:
computing a logistic regression-based detection algorithm to reconstruct and amend a meal record of the user.

Example 91. The non-transitory computer readable medium of example 89 (as well as subject matter in whole or in part of example 90), wherein said extended days are marked “valid” if:
continuous glucose monitoring gaps are no longer than 3 hours;
at least 70% of continuous glucose monitoring data are available; and
at least 2 boluses are recorded. In an embodiment, the monitoring gaps may be any variety as desired or required, such as, but not limited thereto, 30 minutes, 1 hour, 2 hours, etc., or of any combination thereof. In an embodiment, the boluses may be any variety as desired or required, such as, but not limited thereto, 0, 1, or 3, etc.

Example 92. The non-transitory computer readable medium of example 89 (as well as subject matter of one or more of any combination of examples 76-88 and 90-91, in whole or in part), wherein execution of the program instructions by said one or more computer processors cause the one or more processors to:
identify, for each valid extended day, said personalized model of the user's glucose metabolism in the presence of meal ingestion and subcutaneous insulin administration.

Example 93. The non-transitory computer readable medium of example 75 (as well as subject matter of one or more of any combination of examples 76-92, in whole or in part), wherein said personalized model is identified daily. In an embodiment, the identification may be executed as desired or required, such as, but not limited thereto, daily, every 2-3 days, weekly, etc. In an embodiment, the identification may be executed on demand by the user as desired or required.

Example 94. The non-transitory computer readable medium of example 75 (as well as subject matter of one or more of any combination of examples 76-93, in whole or in part), wherein execution of the program instructions by said one or more computer processors cause the one or more processors to:
estimate an additive net-effect signal through model inversion by deconvolution based on said personalized model.

Example 95. The non-transitory computer readable medium of example 90 (as well as subject matter of one or more of any combination of examples 76-89 and 91-94, in whole or in part), wherein the personalized model of the user's glucose metabolism comprises:
identifying a personalized model of the user's glucose metabolism in the presence of meal ingestion and subcutaneous insulin administration; and
estimating an additive net-effect signal through model inversion by deconvolution.

Example 96. The non-transitory computer readable medium of example 94 (as well as subject matter in whole or in part of example 95), wherein execution of the program instructions by said one or more computer processors cause the one or more processors to:
record processed data, model parameters, and net-effect signal in a system database.

Example 97. The non-transitory computer readable medium of example 92 (as well as subject matter of one or more of any combination of examples 76-91 and 93-96, in whole or in part), wherein execution of the program instructions by said one or more computer processors cause the one or more processors to:
periodically replay glucose traces represented by the personalized model of the user's glucose metabolism in a closed-loop fashion with an automated insulin delivery system.

Example 98. The non-transitory computer readable medium of example 97 (as well as subject matter of one or more of any combination of examples 76-96, in whole or in part), wherein execution of the program instructions by said one or more computer processors cause the one or more processors to:
associate said replayed glucose traces with a root mean square error.

Example 99. The non-transitory computer readable medium of example 98 (as well as subject matter of one or more of any combination of examples 76-97, in whole or in part), wherein said glucose traces with a root mean square error above a predefined threshold are excluded from the dataset used to determine insulin-dosing parameters.

Example 100. The non-transitory computer readable medium of example 99 (as well as subject matter of one or more of any combination of examples 76-98, in whole or in part), wherein said predefined threshold is 25 mg/dL. In an embodiment, the predefined threshold may be any variety as desired or required.

Example 101. The non-transitory computer readable medium of example 75 (as well as subject matter of one or more of any combination of examples 76-100, in whole or in part), wherein said insulin-dosing parameters are determined by solving an optimization problem that minimizes the weighted sum of the risks of hypoglycemia (LBGI) and hyperglycemia (HBGI) and the number of hypoglycemic treatments.

Example 102. The non-transitory computer readable medium of example 75 (as well as subject matter of one or more of any combination of examples 76-101, in whole or in part), wherein said insulin-dosing parameters are determined by solving the optimization problem:

$${min}_{\{ {α}_{1} , … , {α}_{6} \} \{ {β}_{1} , … , {β}_{6} \}} J = f ( {BR}^{′} , {CR}^{′} , {CF}^{′} )$$

$$subject to$$

$${α}_{i} ∈ [ - 1 , 1 ] ∀ i = 1 , … , 6$$

$${β}_{i} ∈ [ - 1 , 1 ] ∀ i = 1 , … , 6$$

with α^{v}={α₁, . . . , α₆} and β^{v}={β₁, . . . , β₆] the vector of modulation factors for CR-CF and BR in segments with predefined lengths, and BR′, CR′, and CF′, the modulated profiles through the day for BR and CR-CF computed as a function of α^{v} and β^{v}:

$${BR}^{′} = {BR}_{nom}^{v} \cdot ( 1 + P * \frac{{β}_{ext}^{v}}{100} )$$

$${CR}^{′} = {CR}_{nom}^{v} \cdot ( 1 + P * \frac{{α}_{ext}^{v}}{100} )$$

$${CF}^{′} = {CF}_{nom}^{v} \cdot ( 1 + P * \frac{{α}_{ext}^{v}}{100} )$$

with

$${BR}_{nom}^{v} , {CR}_{nom}^{v} and {CF}_{nom}^{v}$$

the current profiles through the day for BR, CR, and CF in five-minute intervals, and

$${α}_{ext}^{v}$$

and βₑₓₜ^{v} the extended version of α^{v} and β^{v} through the day in five-minute intervals; and
where the cost function (J) is:

$$J = {{\sum }_{j = 1}}^{{N}_{days}} 2 \cdot {LBGI}_{j} + {HBGI}_{j} + {N}_{j , HT}$$

with N_{days} the number of days considered for the optimization (≤14 days), N_{HT} the number of hypoglycemic treatments per day, and LBGIⱼ and HBGIⱼ the hypoglycemia and hyperglycemia indices per day, computed as:

$${Risk}_{k} = log {BG}_{k}^{1.084} - 5.381$$

$${LBGI}_{j} = {{\sum }_{k = 1}}^{{N}_{samples}} 22.77 * {Risk}_{k}^{2} ∀ {Risk}_{k} \leq 0 and {BG}_{k} < 70$$

$${HBGI}_{j} = {{\sum }_{k = 1}}^{{N}_{samples}} 22.77 * {Risk}_{k}^{2} ∀ {Risk}_{k} > 0 and {BG}_{k} < 180$$

with Nₛₐₘₚₗₑₛ=288 the number of samples per day considering a sampling time equal to five minutes. In an embodiment, the number of days considered for the optimization may be any variety as desired or required, such as greater than 14 days.

Example 103. The non-transitory computer readable medium of example 75 (as well as subject matter of one or more of any combination of examples 76-102, in whole or in part), wherein the historical data further includes a finite time range and basal and bolus modulators.

Example 104. The system of example 75 (as well as subject matter of one or more of any combination of examples 76-103, in whole or in part), wherein the said simulation is based on custom modulation factors.

Example 105. The non-transitory computer readable medium of example 104, wherein said custom modulation factors are determined by parsing received historical data into finite time ranges with associated basal and bolus modulators based on said personalized model of the user's glucose metabolism.

Example 106. The non-transitory computer readable medium of example 104 (as well as subject matter in whole or in part of example 105), wherein said custom modulation factors are defined as Pfactors.

Example 107. The non-transitory computer readable medium of example 106 (as well as subject matter of one or more of any combination of examples 76-105, in whole or in part), wherein the said simulation is based on combining optimal modulation factors with said Pfactors to define final modulation profiles for at least one of: basal rate, carbohydrate ratio, and correction factor.

Example 108. The non-transitory computer readable medium of example 75 (as well as subject matter of one or more of any combination of examples 76-107, in whole or in part), wherein said simulation features changes or non-changes to at least one of: basal rate profile, carbohydrate ratio profile, correction factor profiles, or user-specific glucose charts.

Example 109. The non-transitory computer readable medium of example 75 (as well as subject matter of one or more of any combination of examples 76-108, in whole or in part), wherein said treatment includes changes or non-changes to basal and bolus settings.

Example 110. The non-transitory computer readable medium of example 75 (as well as subject matter of one or more of any combination of examples 76-109, in whole or in part), wherein execution of the program instructions by said one or more computer processors cause the one or more processors to:
present the time spent in different glucose ranges via commonly accepted glucose charts.

Example 111. The non-transitory computer readable medium of example 75 (as well as subject matter of one or more of any combination of examples 76-110, in whole or in part), wherein at least part of the historical data is obtained from one or more of the following: an insulin pump device, continuous glucose monitoring device, a manual insulin injection device, a meal-tracking device, and an activity-tracking device.

Example 112. The non-transitory computer readable medium of example 75 (as well as subject matter of one or more of any combination of examples 76-111, in whole or in part), wherein the insulin-dosing parameters are recommended to the user or automatically implemented by a closed-loop control or continuous subcutaneous insulin delivery system.

Example 113. The non-transitory computer readable medium of example 75 (as well as subject matter of one or more of any combination of examples 76-112, in whole or in part), wherein execution of the program instructions by said one or more computer processors cause the one or more processors to perform one or more of the following:
record said plurality of actionable information and risk assessments to a database;
record said plurality of actionable information and risk assessments to a database accessible by the user;
present said plurality of actionable information and risk assessments to the patient via a display system;
present said plurality of actionable information and risk assessments to the user via a website or web application;
record said insulin-dosing parameters based on the personalized model of the user's glucose metabolism to a database;
record said insulin-dosing parameters based on the personalized model of the user's glucose metabolism to a database accessible by the user;
present said insulin-dosing parameters based on the personalized model of the user's glucose metabolism via a display system; and
present said insulin-dosing parameters based on the personalized model of the user's glucose metabolism via a website or web application.

Example 114. A system configured to perform the method of any one or more of Examples 35-74.

Example 115. A non-transitory computer readable medium configured to perform the methods of any one or more of Examples 35-74.

Example 116. The method of using any of the elements, components, devices, computer readable medium and/or systems, or their sub-components, provided in any one or more of examples 1-34 and 75-113, in whole or in part.

Example 117. The method of manufacturing any of the elements, components, devices, computer readable medium and/or systems, or their sub-components, provided in any one or more of examples 1-34 and 75-113, in whole or in part.

### REFERENCES

The devices, systems, apparatuses, modules, compositions, articles of manufacture, materials, computer program products, non-transitory computer readable medium, and methods of various embodiments of the invention disclosed herein may utilize aspects (such as devices, apparatuses, modules, systems, compositions, articles of manufacture, materials, computer program products, non-transitory computer readable medium, and methods) disclosed in the following references, applications, publications and patents and which are hereby incorporated by reference herein in their entirety (and which are not admitted to be prior art with respect to the present invention by inclusion in this section).
1. Thabit H, Hovorka R. Coming of age: the artificial pancreas for type 1 diabetes. Diabetologia. 2016; 59:1795. PMCID: PMC4969330
2. Hinshaw L, Dalla Man C, Nandy D K, Saad A, Bharucha A E, Levine J A, Rizza R A, Basu R, Carter R E, Cobelli C, Kudva Y C, Basu A. Diurnal pattern of insulin action in type 1 diabetes: implications for a closed-loop system. Diabetes. 2013; 62:2223-9. doi: 10.2337/db12-1759. PMCID: PMC3712033
3. Mallad A, Hinshaw L, Dalla Man C, Cobelli C, Basu R, Lingineni R, Carter R E, Kudva Y C, Basu A. Nocturnal glucose metabolism in type 1 diabetes: a study comparing single versus dual tracer approaches. Diabetes Technol Ther. 2015; 17:587-95. doi: 10.1089/dia.2015.0011. PMCID: PMC4528985
4. Hinshaw L, Mallad A, Dalla Man C, Basu R, Cobelli C, Carter R E, Kudva Y C, Basu A. Glucagon sensitivity and clearance in type 1 diabetes: insights from in vivo and in silico experiments. Am J Physiol Endocrinol Metab. 2015; 309: E474-86. doi: 10.1152/ajpendo.00236.2015. PMCID: PMC4556882
5. Visentin R, Dalla Man C, Kudva Y C, Basu A, Cobelli C. Circadian variability of insulin sensitivity: physiological input for in silico artificial pancreas. Diabetes Technol Ther. 2015; 17:1-7. doi: 10.1089/dia.2014.0192. PMCID: PMC4290795
6. Kovatchev B P, Cox D J, Gonder-Frederick L A, Schlundt D, W L Clarke. Stochastic model of self-regulation decision making exemplified by decisions concerning hypoglycemia. Health Psychology. 1998; 17:277-284. PMID: 9619478
7. Kovatchev B P, Cobelli C. Glucose variability: timing, risk analysis, and relationship to hypoglycemia in diabetes. Diabetes Care. 2016; 39:502-510. PMCID: PMC4806774
8. Kovatchev B P, Breton M D, Dalla Man C, Cobelli C. In silico preclinical trials: a proof of concept in closed-loop control of type 1 diabetes. J. Diabetes Sci. Technol. 2009; 3:44-55. PMCID: PMC2681269
9. Basu R, Di Camillo B, Toffolo G, Basu A, Shah P, Vella A, Rizza R, Cobelli C. Use of a novel triple-tracer approach to assess postprandial glucose metabolism. Am J Physiol Endocrinol Metab. 2003; 284: E55-69. PMID: 12485809
10. Basu R, Breda E, Oberg A L, Powell C C, Dalla Man C, Basu A, Vittone J L, Klee G G, Arora P, Jensen M D. Toffolo G, Cobelli C, Rizza R A. Mechanisms of the age-associated deterioration in glucose tolerance: contribution of alterations in insulin secretion, action, and clearance. Diabetes. 2003; 52:1738-48. PMID: 12829641
11. Basu R, Dalla Man C, Campioni M, Basu A, Klee G, Toffolo G, Cobelli C, Rizza R A. Effects of age and sex on postprandial glucose metabolism: differences in glucose turnover, insulin secretion, insulin action, and hepatic insulin extraction. Diabetes. 2006; 55:2001-14. PMID:
12. Nair K S, Rizza R A, O'Brien P, Dhatariya K, Short K R, Nehra A, Vittone J L, Klee G G, Basu A, Basu R, Cobelli C, Toffolo G, Dalla Man C, Tindall D J, Melton L J 3rd, Smith G E, Khosla S, Jensen M D. DHEA in elderly women and DHEA or testosterone in elderly men. N Engl J Med. 2006; 355:1647-59. PMID: 17050889
13. Basu R, Dalla Man C, Campioni M, Basu A, Nair K S, Jensen M D, Khosla S, Klee G, Toffolo G, Cobelli C, Rizza R A. Two years of treatment with dehydroepiandrosterone does not improve insulin secretion, insulin action, or postprandial glucose turnover in elderly men or wornen. Diabetes. 2007; 56:753-66. PMID: 17327446
14. Basu R, Dalla Man C, Campioni M, Basu A, Nair K S, Jensen M D, Khosla S, Klee G, Toffolo G, Cobelli C, Rizza R A. Effect of 2 years of testosterone replacement on insulin secretion, insulin action, glucose effectiveness, hepatic insulin clearance, and postprandial glucose turnover in elderly men. Diabetes Care. 2007; 1972-8. PMID: 17496236
15. Toffolo G, Dalla Man C, Cobelli C, Sunehag A L. Glucose fluxes during OGTT in adolescents assessed by a stable isotope triple tracer method. J Pediatr Endocrinol Metab. 2008; 21:31-45. PMID: 18404971
16. Rizza R A, Toffolo G, Cobelli C. Accurate measurement of postprandial glucose turnover: why is it difficult and how can it be done (relatively) simply?. Diabetes. 2016; 65:1133-45. doi: 10.2337/db15-1166. PMCID: PMC4839208
17. Cobelli C, Dalla Man C, Sparacino G, Magni L,De Nicolao G, Kovatchev B P. Diabetes: models, signals, and control. IEEE Reviews in Biomedical Engineering. 2009; 2:54-96. PMCID: PMC2951686
18. Turksoy K, Bayrak E S, Quinn L, Littlejohn E, Cinar A. Multivariable adaptive closed-loop control of an artificial pancreas without meal and activity announcement. Diabetes Technol Ther. 2013; 15:386-400. PMCID: PMC3643229
19. Dassau E, Pinsker J E, Kudva Y C, Brown S A, Gondhalekar R, Dalla Man C, Patek S, Schiavon M, Dadlani V, Dasanayake I, Church M M, Carter R E, Bevier W C, Huyett L M, Hughes J, Anderson S, Lv D, Schertz E, Emory E, Mccrady-Spitzer S K, Jean T, Bradley P K, Hinshaw L, Laguna Sanz A J, Basu A, Kovatchev B P, Cobelli C, Doyle F J III. 12-week 24/7 ambulatory artificial pancreas with weekly adaptation of insulin delivery settings: effect on hemoglobin A1c and hypoglycemia. Diabetes Care. 2017; 40:1719-1726. PMCID: PMC5711334
20. Shi D, Dassau E, Doyle F J. A Multivariate Bayesian Optimization Framework for Long-Term Controller Adaptation in Artificial Pancreas. 2018 IEEE Conference on Decision and Control (CDC), 2018.
21. Goodwin G. Over One Third of Type 1 Diabetes Patients Quit Closed-Loop System, Endo 2019: The Endocrine Society Annual Meeting . . . me Iscar om/viewarticle/910854
22. Gonder-Frederick L, Cox D, Kovatchev B, Schlundt D, Clarke W. A biopsychobehavioral model of risk of severe hypoglycemia. Diabetes Care. 1997; 20:661-669. PMID: 9097000
23. Cox D J, Gonder-Frederick L A, Kovatchev B P, Young-Hyman D, Schlundt D, Julian D M, Clarke VVL. Bio-psycho-behavioral model of severe hypoglycemia I I: Understanding causes of severe hypoglycemia. Diabetes Care. 1999; 22:2018-2025. PMID: 10587836
24. Clarke W L, Cox D J, Gonder-Frederick L A, Julian D M, Kovatchev B P, Young-Hyman D. The bio-psycho-behavioral model of severe hypoglycemia I I: self-management behaviors. Diabetes Care. 1999; 22:580-584. PMID: 10189535
25. Cox D J, Carter W R, Gonder-Frederick L A, Clarke W L, Pohl S L. Blood glucose discrimination training in insulin-dependent diabetes mellitus (IDDM) patients. Biofeedback Self Regul. 1988; 13:201-PMID: 3228550
26. Cox D J, Gonder-Frederick L A, Lee J H, Julian D M, Carter W R, Clarke W L. Effects and correlates of blood glucose awareness training among patients with IDDM. Diabetes Care. 1989; 12:313-8. PMID: 2721340
27. Cox D J, Gonder-Frederick L, Julian D, Cryer P, Lee J H, Richards F E, Clarke W. Intensive versus standard blood glucose awareness training (BGAT) with insulin-dependent diabetes: mechanisms and ancillary effects. Psychosomatic Medicine. 1991; 53:453-62. PMID: 1924656
28. Cox D J, Gonder-Frederick L, Julian D M, Clarke W. Long-term follow-up evaluation of blood glucose awareness training. Diabetes Care. 1994; 17:1-5. PMID: 8112183
29. Cox D J, Gonder-Frederick L A, Polonsky W, Schlundt D, Julian D, Clarke W L. A multicenter evaluation of blood glucose awareness training-I I. Diabetes Care. 1995; 18:523-528. PMID: 7497863
30. Cox D J, Gonder-Frederick L A, Polonsky W, Schlundt D, Julian D, Kovatchev B P, Clarke W L. Blood glucose awareness training (BGAT-I I): long term benefits. Diabetes Care. 2001; 24:637-642. PMID: 11315822
31. Cox D J, Gonder-Frederick L, Ritterband L, Patel K, Schächinger H, Fehm-Wolfsdorf G, Hermanns N, Snoek F, Zrebiec J, Polonsky W, Schlundt D. Blood glucose awareness training: what is it, where is it, and where is it going?. Diabetes Spectrum. 2006; 19:43-9. doi: 10.2337/diaspect. 19.1.43
32. Cox D, Ritterband L, Magee J, Clarke W, Gonder-Frederick L. Blood glucose awareness training delivered over the internet. Diabetes Care. 2008; 31:1527-8. PMCID: PMC2494647
33. Broers S, Le Cessie S, Van Vliet K P, Spinhoven P, Van Der Ven N C, Radder J K. Blood glucose awareness training in Dutch type 1 diabetes patients. Short-term evaluation of individual and group training. Diabetic Medicine. 2002; 19:157-61. PMID: 11874433
34. Broers S, van Vliet K P, le Cessie S, Spinhoven P, van der Ven N C, Radder J K. Blood glucose awareness training in Dutch type 1 diabetes patients: one-year follow-up. Neth J Med. 2005; 63:164-169. PMID: 15952484
35. Fehm-Wolfsdorf G, Peters A. Effectiveness of BGAT in Germany (Erfolgreiche Arbeit mit dem Blutglukose-Wahrnehmungstraining (BGAT) in deutschen Praxen und Kliniken). Diabetes und Stoffwechsel4: 116.
36. Grossman A, Barenboim E, Azaria B, Goldstein L, Cohen O. Blood glucose awareness training helps return insulin-treated aviators to the cockpit. Aviat Space Environ Med. 2005; 76:586-8. PMID: 15945405
37. Hermanns N, Kulzer B, Kubiak T, Krichbaum M, Haak T. The effect of an education programme (HyPOS) to treat hypoglycaemia problems in patients with type 1 diabetes. Diabetes/metabolism Research and Reviews. 2007; 23:528-38. PMID: 17245692
38. Hermanns N, Kulzer B, Krichbaum M, Kubiak T, Haak T. Long-term effect of an education program (HyPOS) on the incidence of severe hypoglycemia in patients with type 1 diabetes. Diabetes Care. 2010; 33: e36. PMID: 20190284
39. Kinsley B T, Weinger K, Bajaj M, Levy C J, Simonson D C, Quigley M, Cox D J, Jacobson A M. Blood glucose awareness training and epinephrine responses to hypoglycemia during intensive treatment in type 1 diabetes. Diabetes Care. 1999; 22:1022-8. PMID: 10388961
40. Nurick M A, Johnson S B. Enhancing blood glucose awareness in adolescents and young adults with IDDM. Diabetes Care 1991; 14:1-7. PMID: 1991430
41. Rondags S M, de Wit M, van Tulder M W, Diamant M, Snoek F J. HypoAware-a brief and partly web-based psycho-educational group intervention for adults with type 1 and insulin-treated type 2 diabetes and problematic hypoglycaemia: design of a cost-effectiveness randomised controlled trial. BMC Endocrine Disorders. 2015; 15:43. PMCID: PMC4546185
42. Schachinger H, Hegar K, Hermanns N, Straumann M, Keller U, Fehm-Wolfsdorf G, Berger W, Cox D. Randomized controlled clinical trial of blood glucose awareness training (BGAT III) in Switzerland and Germany. Journal of Behavioral Medicine. 2005; 28:587-94. PMID: 16222412
43. Snoek F J, Van Der Ven N C, Twisk J W, Hogenelst M H, Tromp-Wever A M, Van Der Ploeg H M, Heine R J. Cognitive behavioural therapy (CBT) compared with blood glucose awareness training (BGAT) in poorly controlled type 1 diabetic patients: long-term effects on HbA1c moderated by depression. A randomized controlled trial. Diabetic Medicine. 2008; 25:1337-42. PMID: 19046225
44. ter Braak E W, de Valk H W, de la Bije Y F, van der Laak M F, van Haeften T W, Erkelens D W. Response to training in blood glucose awareness is related to absence of previous hypoglycemic coma. Diabetes Care. 2000; 23:1199-1200. PMID: 10937524
45. van der Ven NCW, Lubach CHC, Hogenelst, MHE, van Iperen A, Tromp-Wever AME, Vriend A, van der Ploeg H M, Heine R J, Snoek F J. Cognitive behavioral group training (CBGT) for patients with type 1 diabetes in persistent poor glycaemic control: who do we reach? Patient Educ Couns. 2005; 56:313-322. PMID: 15721974
46. van der Ven NCW, Hogenelst MHE, Tromp-Wever AME, Twisk J, Ploeg H M van der, Heine R J, Snoek F J. Short-term effects of cognitive-behavioural group training (CBGT) in adults with type 1 diabetes in prolonged poor glycaemic control: a randomized controlled trial. Diabet Med. 2005; 22:1619-23. PMID: 16241932
47. Veneman T F. Diabetes mellitus and traffic incidents. The Netherlands J Medicine. 1996; 48:2427-628. PMID: 8775749
48. Gonder-Frederick L A, Grabman J H, Kovatchev B, Brown S A, Patek S, Basu A, Pinsker J E, Kudva Y C, Wakeman C A, Dassau E, Cobelli C. Is psychological stress a factor for incorporation into future closed-loop systems? Journal of Diabetes Science and Technology. 2016; 10 (3): 640-PMCID: PMC5038545
49. Barnard K D, Lloyd C, Dyson P, Davies M J, O'Neill S, Kanumilli N, Lawton J, Ziegler R, Holt RIG. Kaleidoscope model of diabetes care: time for a rethink? Diabetic Medicine. 2014; 31:522-30. PMID: 24506524
50. Barnard K D, Hitchcock J, Hood K K, et al. KALMOD: novel holistic clinical tool to improve consultations and outcomes. Diabetes. 2017; 66 (Suppl 1A): 89.
51. Gonzalez, J. S., Tanenbaum, M. L., & Commissariat, P. V. Psychosocial factors in medication adherence and diabetes self-management: Implications for research and practice. American Psychologist, 2016; 71:539-551. PMID: 27690483, PMCID: PMC5792162.
52. Kovatchev B P, Cobelli C. Glucose variability: timing, risk analysis, and relationship to hypoglycemia in diabetes. Diabetes Care. 2016; 39:502-510. PMCID: PMC4806774
53. Kovatchev B P, Anderson S M, Raghinaru D, et al, for the IDCL Trial Research Group. Randomized Controlled Trial of Mobile Closed-Loop Control. Diabetes Care. 2020; 43:607-615. PMID: 31937608
54. Brown S A, Kovatchev B P, Raghinaru D, et al, for the IDCL Trial Research Group. 6-month randomized multicenter trial of closed-loop control in type 1 diabetes. N Engl J Med. 2019; 381:1707-17. PMID: 31618560.
55. Breton M D, Kanapka L G, Beck R W, et al, for the IDCL Trial Research Group. A Randomized Trial of Closed-Loop Control in Children with Type 1 Diabetes. N Engl J Med. 2020; 383:836-845. PMID: 32846062.

### ADDITIONAL REFERENCES

The devices, systems, apparatuses, modules, compositions, articles of manufacture, materials, computer program products, non-transitory computer readable medium, and methods of various embodiments of the invention disclosed herein may utilize aspects (such as devices, apparatuses, modules, systems, compositions, articles of manufacture, materials, computer program products, non-transitory computer readable medium, and methods) disclosed in the following references, applications, publications and patents and which are hereby incorporated by reference herein in their entirety (and which are not admitted to be prior art with respect to the present invention by inclusion in this section).

A. U.S. Utility patent application Ser. No. 17/679,265, entitled “METHOD AND SYSTEM OF FULLY-AUTOMATED ARTIFICIAL PANCREAS CONTROL FOR COUNTERACTING POSTPRANDIAL HYPERGLYCEMIA”, filed Feb. 24, 2022; Publication No. US 2022-0273874 A1, Sep. 1, 2022.

B. U.S. Utility patent application Ser. No. 17/611,323, entitled “SYSTEM AND METHOD FOR ARTIFICIAL PANCREAS WITH MULTI-STAGE MODEL PREDICTIVE CONTROL”, filed Nov. 15, 2021; Publication No. US 2022-0203029 A1, Jun. 30, 2022.

C. International Patent Application Serial No. PCT/US2020/032855, entitled “SYSTEM AND METHOD FOR ARTIFICIAL PANCREAS WITH MULTI-STAGE MODEL PREDICTIVE CONTROL”, filed May 14, 2020; Publication No. WO 2020/232232, Nov. 19, 2020.

D. International Patent Application Serial No. PCT/US2022/043651, entitled “USING AN ONLINE DISTURBANCE REJECTION AND ANTICIPATION SYSTEM TO REDUCE HYPERGLYCEMIA”, filed Sep. 15, 2022; Publication No. WO2023043915, Mar. 23, 2023.

E. U.S. Utility patent application Ser. No. 17/932,496, entitled “USING AN ONLINE DISTURBANCE REJECTION AND ANTICIPATION SYSTEM TO REDUCE HYPERGLYCEMIA”, filed Sep. 15, 2022; Publication No. US-2023-0097161-A1, Mar. 30, 2023.

F. U.S. Utility patent application Ser. No. 17/694,870, entitled “INSULIN MONITORING AND DELIVERY SYSTEM AND METHOD FOR CGM BASED FAULT DETECTION AND MITIGATION VIA METABOLIC STATE TRACKING”, filed Mar. 15, 2022; Publication No. US 2022-0203020 A1, Jun. 30, 2022.

G. U.S. Utility patent application Ser. No. 15/580,935, entitled “INSULIN MONITORING AND DELIVERY SYSTEM AND METHOD FOR CGM BASED FAULT DETECTION AND MITIGATION VIA METABOLIC STATE TRACKING”, filed Dec. 8, 2017; U.S. Pat. No. 11,311,665, issued Apr. 26, 2022; Publication No. US-2019-0254595-A1, Aug. 22, 2019.

H. International Patent Application Serial No. PCT/US2016/036729, entitled “INSULIN MONITORING AND DELIVERY SYSTEM AND METHOD FOR CGM BASED FAULT DETECTION AND MITIGATION VIA METABOLIC STATE TRACKING”, filed Jun. 9, 2016; Publication No. WO 2016/201120, Dec. 15, 2016.

I. U.S. Utility patent application Ser. No. 18/041,659, entitled “METHOD AND SYSTEM FOR GENERATING A USER TUNABLE REPRESENTATION OF GLUCOSE HOMEOSTASIS IN TYPE 1 DIABETES BASED ON AUTOMATED RECEIPT OF THERAPY PROFILE DATA”, filed Feb. 14, 2023; Publication No. 20230352185, Nov. 2, 2023.

J. International Patent Application Serial No. PCT/US2021/045936, entitled “METHOD AND SYSTEM FOR GENERATING A USER TUNABLE REPRESENTATION OF GLUCOSE HOMEOSTASIS IN TYPE 1 DIABETES BASED ON AUTOMATED RECEIPT OF THERAPY PROFILE DATA”, filed Aug. 13, 2021; Publication No. WO 2022/036214, Feb. 17, 2022.

K. U.S. Utility patent application Ser. No. 18/031,976, entitled “METHOD AND SYSTEM OF CLOSED LOOP CONTROL IMPROVING GLYCEMIC RESPONSE FOLLOWING AN UNANNOUNCED SOURCE OF GLYCEMIC FLUCTUATION”, filed Apr. 14, 2023; Publication No. 20240024576, Jan. 25, 2024.

L. International Patent Application Serial No. PCT/US2021/054894, entitled “METHOD AND SYSTEM OF CLOSED LOOP CONTROL IMPROVING GLYCEMIC RESPONSE FOLLOWING AN UNANNOUNCED SOURCE OF GLYCEMIC FLUCTUATION”, filed Oct. 14, 2021; Publication No. WO 2022/081788, Apr. 21, 2022.

M. International Patent Application Serial No. PCT/US2023/078434, entitled “SYSTEM AND METHOD FOR DETECTING SENSOR COMPRESSION OF CONTINUOUS GLUCOSE MONITORING (CGM) SENSORS”, filed Nov. 2, 2023.

N. U.S. Utility patent application Ser. No. 18/500,403, entitled “SYSTEM AND METHOD FOR DETECTING SENSOR COMPRESSION OF CONTINUOUS GLUCOSE MONITORING (CGM) SENSORS”, filed Nov. 2, 2023.

O. International Patent Application Serial No. PCT/US2023/020014, entitled “System and Method for Identifying Clinically-Similar Clusters of Daily Continuous Glucose Monitoring (CGM) Profiles”, filed Apr. 26, 2023; Publication No. WO/2023/212076, Nov. 2, 2023.

P. International Patent Application Serial No. PCT/US2023/078441, entitled“ ”, filed Nov. 2, 2023.

Q. U.S. Utility patent application Ser. No. 18/500,212, entitled “SYSTEM AND METHOD FOR DETECTING PRESSURE INDUCED SENSOR ATTENUATIONS OF CONTINUOUS GLUCOSE MONITORING (CGM)”, filed Nov. 2, 2023.

R. International Patent Application Serial No. PCT/US2023/037178, entitled “METHOD AND SYSTEM OF PENALIZATION FOR MODEL PREDICTIVE CONTROL IN AUTOMATED INSULIN DELIVERY”, filed Nov. 13, 2023.

S. U.S. Utility patent application Ser. No. 18/507,253, entitled “METHOD AND SYSTEM OF PENALIZATION FOR MODEL PREDICTIVE CONTROL IN AUTOMATED INSULIN DELIVERY”, filed Nov. 13, 2023.

T. International Patent Application Serial No. PCT/US2023/015169, entitled “COMBINATION SODIUM-GLUCOSE COTRANSPORTER INHIBITOR (SGLTi)-INSULIN THERAPY FOR GLYCEMIC CONTROL IN TYPE 1 DIABETES”, filed Mar. 14, 2023; Publication No. WO2023177654, Sep. 21, 2023.

U. International Patent Application Serial No. PCT/US2023/024301, entitled “System, Method, and Computer Readable Medium for CGM-based OGTT Replacement Diagnosis Test”, filed Jun. 2, 2023; Publication No. WO/2023/235573, Dec. 7, 2023.

V. U.S. Utility patent application Ser. No. 29/467,039, entitled “Alarm Clock Display of Personal Blood Glucose Level”, filed Sep. 13, 2013.

W. U.S. Utility patent application Ser. No. 17/132,604, entitled “ACCURACY CONTINUOUS GLUCOSE MONITORING METHOD, SYSTEM, AND DEVICE”, filed Dec. 23, 2020; Publication No. US 2021-0113122 A1, Apr. 22, 2021.

X. U.S. Utility patent application Ser. No. 15/510,878, entitled “ACCURACY CONTINUOUS GLUCOSE MONITORING METHOD, SYSTEM, AND DEVICE”, filed Mar. 13, 2017; U.S. Pat. No. 10,881,334, issued Jan. 5, 2021; Publication No. US-2017-0273607-A1, Sep. 28, 2017.

Y. International Patent Application Serial No. PCT/US2015/045340, entitled “IMPROVED ACCURACY CONTINUOUS GLUCOSE MONITORING METHOD, SYSTEM, AND DEVICE”, filed Aug. 14, 2015; Publication No. WO2016025874, Feb. 18, 2016.

Z. U.S. Utility patent application Ser. No. 13/393,647, entitled “System, Method and Computer Program Product for Adjustment of Insulin Delivery (AID) in Diabetes Using Nominal Open-Loop Profiles”, filed Mar. 1, 2012; Publication No. 2012/0245556, Sep. 27, 2012.

AA. International Patent Application Serial No. PCT/US2010/047386, entitled “System, Method and Computer Program Product for Adjustment of Insulin Delivery (AID) in Diabetes Using Nominal Open-Loop Profiles”, filed Aug. 31, 2010; Publication No. WO 2011/028731, Mar. 10, 2011.

BB. U.S. Utility patent application Ser. No. 18/325,564, entitled “METHOD, SYSTEM AND COMPUTER PROGRAM PRODUCT FOR CGM-BASED PREVENTION OF HYPOGLYCEMIA VIA HYPOGLYCEMIA RISK ASSESSMENT AND SMOOTH REDUCTION INSULIN DELIVERY”, filed May 30, 2023; Publication No. US2023-0293062A1, Sep. 21, 2023.

CC. U.S. Utility patent application Ser. No. 18/323,117, entitled “METHOD, SYSTEM AND COMPUTER PROGRAM PRODUCT FOR CGM-BASED PREVENTION OF HYPOGLYCEMIA VIA HYPOGLYCEMIA RISK ASSESSMENT AND SMOOTH REDUCTION INSULIN DELIVERY”, filed May 24, 2023; Publication No. US 2024-0023837 A1, Jan. 25, 2024.

DD. U.S. Utility patent application Ser. No. 18/069,551, entitled “METHOD, SYSTEM AND COMPUTER PROGRAM PRODUCT FOR CGM-BASED PREVENTION OF HYPOGLYCEMIA VIA HYPOGLYCEMIA RISK ASSESSMENT AND SMOOTH REDUCTION INSULIN DELIVERY”, filed Dec. 21, 2022; U.S. Pat. No. 11,751,779, issued Sep. 12, 2023; Publication No. 20230125221, Apr. 27, 2023.

EE. U.S. Utility patent application Ser. No. 17/333,161, entitled “METHOD, SYSTEM AND COMPUTER PROGRAM PRODUCT FOR CGM-BASED PREVENTION OF HYPOGLYCEMIA VIA HYPOGLYCEMIA RISK ASSESSMENT AND SMOOTH REDUCTION INSULIN DELIVERY”, filed May 28, 2021; Publication No. US 2021-0282677 A1, Sep. 16, 2021.

FF. U.S. Utility patent application Ser. No. 17/070,245, entitled “METHOD, SYSTEM AND COMPUTER PROGRAM PRODUCT FOR CGM-BASED PREVENTION OF HYPOGLYCEMIA VIA HYPOGLYCEMIA RISK ASSESSMENT AND SMOOTH REDUCTION INSULIN DELIVERY”, filed Oct. 14, 2020; U.S. Pat. No. 11,723,562, issued Aug. 15, 2023; Publication No. US 2021/0038132 A1, Feb. 11, 2021.

GG. U.S. Utility patent application Ser. No. 15/669,111, entitled “METHOD, SYSTEM AND COMPUTER PROGRAM PRODUCT FOR CGM-BASED PREVENTION OF HYPOGLYCEMIA VIA HYPOGLYCEMIA RISK ASSESSMENT AND SMOOTH REDUCTION INSULIN DELIVERY”, filed Aug. 4, 2017; U.S. Pat. No. 10,842,419, issued Nov. 24, 2020; Publication No. US-2017-0337348-A1, Nov. 23, 2017.

HH. U.S. Utility patent application Ser. No. 14/015,831, entitled “CGM-Based Prevention of Hypoglycemia Via Hypoglycemia Risk Assessment and Smooth Reduction of Insulin Delivery”, filed Aug. 30, 2013; U.S. Pat. No. 9,750,438, issued Sep. 5, 2017; Publication No. 20140046159, Feb. 13, 2014.

II. U.S. Utility patent application Ser. No. 13/203,469, entitled “CGM-Based Prevention of Hypoglycemia via Hypoglycemia Risk Assessment and Smooth Reduction Insulin Delivery”, filed Aug. 25, 2011; U.S. Pat. No. 8,562,587, issued Oct. 22, 2013; Publication No. 2012/0059353, Mar. 8, 2012.

JJ. International Patent Application Serial No. PCT/US2010/025405, entitled “CGM-BASED PREVENTION OF HYPOGLYCEMIA VIA HYPOGLYCEMIA RISK ASSESSMENT AND SMOOTH REDUCTION INSULIN DELIVERY”, filed Feb. 25, 2010; Publication No. WO 2010/099313 A1, Sep. 2, 2010.

KK. U.S. Utility patent application Ser. No. 17/776,717, entitled “METHOD AND SYSTEM FOR MODEL-BASED TRACKING OF HEMOGLOBIN A1c FROM DAILY CONTINUOUS GLUCOSE MONITORING PROFILES”, filed May 13, 2022; Publication No. 20220395200, Dec. 15, 2022.

LL. International Patent Application Serial No. PCT/US2020/060658, entitled “METHOD AND SYSTEM FOR MODEL-BASED TRACKING OF HEMOGLOBIN A1c FROM DAILY CONTINUOUS GLUCOSE MONITORING PROFILES”, filed Nov. 16, 2020; Publication No. WO 2021/097396, May 20, 2021.

MM. U.S. Utility patent application Ser. No. 14/799,329, entitled “ACCURACY OF CONTINUOUS GLUCOSE SENSORS”, filed Jul. 14, 2015; U.S. Pat. No. 10,194,850, issued Feb. 5, 2019; Publication No. US-2016-0007890-A1, Jan. 14, 2016.

NN U.S. Utility patent application Ser. No. 12/065,257, entitled “Accuracy of Continuous Glucose Sensors”, filed Feb. 28, 2008; Publication No. 2008/0314395, Dec. 25, 2008 00. International Patent Application Serial No. PCT/US2006/033724, entitled “Method for Improvising Accuracy of Continuous Glucose Sensors and a Continuous Glucose Sensor Using the Same”, filed Aug. 29, 2006; Publication No. WO07027691, Mar. 8, 2007.

PP. U.S. Utility patent application Ser. No. 16/126,879, entitled “Method, System and Computer Program Product for Evaluation of Insulin Sensitivity, Insulin/Carbohydrate Ratio, and Insulin Correction Factors in Diabetes from Self-Monitoring Data”, filed Sep. 10, 2018; Publication No. US-2019-0019571-A1, Jan. 17, 2019.

QQ. U.S. Utility patent application Ser. No. 12/665,149, entitled “Method, System and Computer Program Product for Evaluation of Insulin Sensitivity, Insulin/Carbohydrate Ratio, and Insulin Correction Factors in Diabetes from Self-Monitoring Data”, filed Dec. 17, 2009; Publication No. 2010/0198520, Aug. 5, 2010.

RR. International Patent Application Serial No. PCT/US2008/069416, entitled “Method, System and Computer Program Product for Evaluation of Insulin Sensitivity, Insulin/Carbohydrate Ratio, and Insulin Correction Factors in Diabetes from Self-Monitoring Data”, filed Jul. 8, 2008; Publication No. WO 2009/009528, Jan. 15, 2009.

SS. U.S. Utility patent application Ser. No. 12/159,891, entitled “Method, System and Computer Program Product for Evaluation of Blood Glucose Variability in Diabetes from Self-Monitoring Data”, filed Jul. 2, 2008; U.S. Pat. No. 11,355,238, issued Jun. 7, 2022; Publication No. 2009/0171589, Jul. 2, 2009.

TT. International Patent Application Serial No. PCT/US2007/000370, entitled “Method, System and Computer Program Product for Evaluation of Blood Glucose Variability in Diabetes from Self-Monitoring Data”, filed Jan. 5, 2007; Publication No. WO07081853, Jul. 19, 2007.

UU. U.S. Utility patent application Ser. No. 16/789,901, entitled “Unified Platform for Monitoring and Control of Blood Glucose Levels in Diabetic Patients”, filed Feb. 13, 2020; Publication No. US-2020-0214629-A1, Jul. 9, 2020.

VV. U.S. Utility patent application Ser. No. 14/128,922, entitled “Unified Platform for Monitoring and Control of Blood Glucose Levels in Diabetic Patients”, filed Dec. 23, 2013; U.S. Pat. No. 10,610,154, issued Apr. 7, 2020; Publication No. 2015/0018633, Jan. 15, 2015.

WW. International Patent Application Serial No. PCT/US2012/043910, entitled “Unified Platform for Monitoring and Control of Blood Glucose Levels in Diabetic Patients”, filed Jun. 23, 2012; Publication No. WO 2012/178134, Dec. 27, 2012.

XX U.S. Utility patent application Ser. No. 17/181,888, entitled “SYSTEMS OF CENTRALIZED DATA EXCHANGE FOR MONITORING AND CONTROL OF BLOOD GLUCOSE”, filed Feb. 22, 2021; Publication No. US 2021-0169409 A1, Jun. 10, 2021.

YY. U.S. Utility patent application Ser. No. 15/109,682, entitled “SYSTEMS OF CENTRALIZED DATA EXCHANGE FOR MONITORING AND CONTROL OF BLOOD GLUCOSE”, filed Jul. 5, 2016; U.S. Pat. No. 10,925,536, issued Feb. 23, 2021; Publication No. US-2016-0331310-A1, Nov. 17, 2016.

ZZ. International Patent Application Serial No. PCT/US2015/010167, entitled “Central Data Exchange Node for System Monitoring and Control of Blood Glucose Levels in Diabetic Patients”, filed Jan. 5, 2015; Publication No. WO2015103543, Jul. 9, 2015.

AAA. U.S. Utility patent application Ser. No. 11/578,831, entitled “Method, System and Computer Program Product for Evaluating the Accuracy of Blood Glucose Monitoring Sensors/Devices”, filed Oct. 18, 2006; U.S. Pat. No. 7,815,569, issued Oct. 19, 2010; Publication No. 2007/0232878, Oct. 4, 2007.

BBB. International Patent Application Serial No. US2005/013792, entitled “Method, System and Computer Program Product for Evaluating the Accuracy of Blood Glucose Monitoring Sensors/Devices”, filed Apr. 21, 2005; Publication No. WO05106017, Nov. 10, 2005.

CCC. U.S. Utility patent application Ser. No. 17/156,169, entitled “Method, System and Computer Program Product for Real-Time Detection of Sensitivity Decline in Analyte Sensors”, filed Jan. 22, 2021; U.S. Pat. No. 11,722,229, issued Aug. 8, 2023; Publication No. US 2021-0218481 A1, Jul. 15, 2021.

DDD. U.S. Utility patent application Ser. No. 15/866,384, entitled “Method, System and Computer Program Product for Real-Time Detection of Sensitivity Decline in Analyte Sensors”, filed Jan. 9, 2018; U.S. Pat. No. 10,903,914, issued Jan. 26, 2021; Publication No. US-2018-0323882-A1, Nov. 8, 2018.

EEE. U.S. Utility patent application Ser. No. 14/266,612, entitled “Method, System and Computer Program Product for Real-Time Detection of Sensitivity Decline in Analyte Sensors”, filed Apr. 30, 2014; U.S. Pat. No. 9,882,660, issued Jan. 30, 2018; Publication No. 2014/0244216, Aug. 28, 2014.

FFF. U.S. Utility patent application Ser. No. 13/418,305, entitled “Method, System and Computer Program Product for Real-Time Detection of Sensitivity Decline in Analyte Sensors”, filed Mar. 12, 2012; U.S. Pat. No. 8,718,958, issued May 6, 2014; Publication No. 2012/0173200, Jul. 5, 2012.

GGG. International Patent Application Serial No. PCT/US2007/082744, entitled “Method, System and Computer Program Product for Real-Time Detection of Sensitivity Decline in Analyte Sensors”, filed Oct. 26, 2007; Publication No. WO/2008/052199, May 2, 2008.

HHH. U.S. Utility patent application Ser. No. 11/925,689, entitled “Method, System and Computer Program Product for Real-Time Detection of Sensitivity Decline in Analyte Sensors”, filed Oct. 26, 2007; U.S. Pat. No. 8,135,548, issued Mar. 13, 2012; Publication No. 2008/0172205, Jul. 17, 2008.

III. U.S. Utility patent application Ser. No. 12/975,580, entitled “Method, System, and Computer Program Product for the Evaluation of Glycemic Control in Diabetes from Self-Monitoring Data”, filed Dec. 22, 2010; Publication No. 2012/0004512, Jan. 5, 2012.

JJJ. U.S. Utility patent application Ser. No. 11/305,946, entitled “Method, System, and Computer Program Product for the Evaluation of Glycemic Control in Diabetes from Self-Monitoring Data”, filed Dec. 19, 2005; U.S. Pat. No. 7,874,985, issued Jan. 25, 2011; Publication No. 2006/0094947, May 4, 2006.

KKK. U.S. Utility patent application Ser. No. 10/240,228, entitled “Method, System, and Computer Program Product for the Evaluation of Glycemic Control in Diabetes from Self-Monitoring Data”, filed Sep. 26, 2002; U.S. Pat. No. 7,025,425, issued Apr. 11, 2006; Publication No. 2003/0212317, Nov. 13, 2003.

LLL. International Patent Application Serial No. PCT/US2001/009884, entitled “Method, System, and Computer Program Product for the Evaluation of Glycemic Control in Diabetes”, filed Mar. 29, 2001; Publication No. WO 01/72208, Oct. 4, 2001.

MMM. U.S. Utility patent application Ser. No. 10/524,094, entitled “Method, System, And Computer Program Product for The Processing of Self-Monitoring Blood Glucose (SMBG) Data To Enhance Diabetic Self-Management”, filed Feb. 9, 2005; U.S. Pat. No. 8,538,703, issued Sep. 17, 2013; Publication No. 2005214892, Sep. 29, 2005.

NNN. International Patent Application Serial No. PCT/US2003/025053, entitled “Managing and Processing Self-Monitoring Blood Glucose”, filed Aug. 8, 2003; Publication No. WO 2004/015539, Feb. 19, 2004.

000. U.S. Utility patent application Ser. No. 16/451,766, entitled “TRACKING CHANGES IN AVERAGE GLYCEMIA IN DIABETICS”, filed Jun. 25, 2019; U.S.

U.S. Pat. No. 11,842,817, issued Dec. 12, 2023; Publication No. US-2019-0318801-A1, Oct. 17, 2019.

PPP. U.S. Utility patent application Ser. No. 14/769,638, entitled “METHOD AND SYSTEM FOR MODEL-BASED TRACKING OF CHANGES IN AVERAGE GLYCEMIA IN DIABETES”, filed Aug. 21, 2015; U.S. Pat. No. 10,332,615, issued Jun. 25, 2019; Publication No. US-2016-0004813-A1, Jan. 7, 2016.

QQQ. International Patent Application Serial No. PCT/US2014/017754, entitled “METHOD AND SYSTEM FOR MODEL-BASED TRACKING OF CHANGES IN AVERAGE GLYCEMIA IN DIABETES”, filed Feb. 21, 2014; Publication No. WO 2014/130841, Aug. 28, 2014.

RRR. U.S. Utility patent application Ser. No. 15/580,915, entitled “System and Method for Tracking Changes in Average Glycemia in Diabetics”, filed Dec. 8, 2017; U.S. Pat. No. 11,567,062, issued Jan. 31, 2023; Publication No. US-2018-0313815-A1, Nov. 1, 2018.

SSS. International Patent Application Serial No. PCT/US2016/036481, entitled “System and Method for Tracking Changes in Average Glycemia in Diabetics”, filed Jun. 8, 2016; Publication No. WO2016200970, Dec. 15, 2016.

TTT. U.S. Utility patent application Ser. No. 12/516,044, entitled “Method, System, and Computer Program Product for the Detection of Physical Activity by Changes in Heart Rate, Assessment of Fast Changing Metabolic States, and Applications of Closed and Open Control Loop in Diabetes”, filed May 22, 2009; U.S. Pat. No. 8,585,593, issued Nov. 19, 2013; Publication No. 2010/0057043, Mar. 4, 2010.

UUU. International Patent Application Serial No. PCT/US2007/085588, entitled “Method, System, and Computer Program Product for the Detection of Physical Activity by Changes in Heart Rate, Assessment of Fast Changing Metabolic States, and Applications of Closed and Open Control Loop in Diabetes”, filed Nov. 27, 2007; Publication No. WO2008/067284, Jun. 5, 2008.

VVV. U.S. Utility patent application Ser. No. 16/588,881, entitled “Tracking the Probability for Imminent Hypoglycemia in Diabetes from Self-Monitoring Blood Glucose (SMBG) Data”, filed Sep. 30, 2019; Publication No. US-2020-0066410-A1, Feb. 27, 2020.

WWW. U.S. Utility patent application Ser. No. 13/394,091, entitled “Tracking the Probability for Imminent Hypoglycemia in Diabetes from Self-Monitoring Blood Glucose (SMBG) Data”, filed Mar. 2, 2012; U.S. Pat. No. 10,431,342, issued Oct. 1, 2019; Publication No. 2012/0191361, Jul. 26, 2012.

XXX. International Patent Application Serial No. PCT/US2010/047711, entitled “Tracking the Probability for Imminent Hypoglycemia in Diabetes from Self-Monitoring Blood Glucose (SMBG) Data”, filed Sep. 2, 2010; Publication No. WO 2011/028925, Mar. 10, 2011.

YYY. U.S. Utility patent application Ser. No. 17/683,676, entitled “System, Method and Computer Readable Medium for Dynamical Tracking of the Risk for Hypoglycemia in Type 1 and Type 2 Diabetes”, filed Mar. 1, 2022; Publication No. US 2022-0262519 A1, Aug. 18, 2022.

ZZZ. U.S. Utility patent application Ser. No. 15/958,257, entitled “System, Method and Computer Readable Medium for Dynamical Tracking of the Risk for Hypoglycemia in Type 1 and Type 2 Diabetes”, filed Apr. 20, 2018; U.S. Pat. No. 11,289,201, issued Mar. 29, 2022; Publication No. US-2018-0366223-A1, Dec. 20, 2018.

AAAA. International Patent Application Serial No. PCT/US2016/058234, entitled “System, Method and Computer Readable Medium for Dynamical Tracking of the Risk for Hypoglycemia in Type 1 and Type 2 Diabetes”, filed Oct. 21, 2016; Publication No. WO 2017/070553, Apr. 27, 2017.

BBBB. U.S. Utility patent application Ser. No. 11/943,226, entitled “Systems, Methods and Computer Program Codes for Recognition of Patterns of Hyperglycemia and Hypoglycemia, Increased Glucose Variability, and Ineffective Self-Monitoring in Diabetes”, filed Nov. 20, 2007; Publication No. 2008/0154513, Jun. 26, 2008.

CCCC. U.S. Utility patent application Ser. No. 14/128,811, entitled “Methods and Apparatus for Modular Power Management and Protection of Critical Services in Ambulatory Medical Devices”, filed Dec. 23, 2013; U.S. Pat. No. 9,430,022, issued Aug. 30, 2016; Publication No. 2014/0215239, Jul. 31, 2014.

DDDD. International Patent Application Serial No. PCT/US2012/043883, entitled “Methods and Apparatus for Modular Power Management and Protection of Critical Services in Ambulatory Medical Devices”, filed Jun. 22, 2012; Publication No. WO 2012/178113, Dec. 27, 2012.

EEEE. International Patent Application Serial No. PCT/US2022/032121, entitled “METHOD FOR STRUCTURING AND CLASSIFICATION OF CONTINUOUS GLUCOSE MONITORING (CGM) PROFILES”, filed Jun. 3, 2022; Publication No. WO2022256626, Dec. 8, 2022.

FFFF. U.S. Utility patent application Ser. No. 17/829,754, entitled “METHOD FOR STRUCTURING AND CLASSIFICATION OF CONTINUOUS GLUCOSE MONITORING (CGM) PROFILES”, filed Jun. 1, 2022; Publication No. 20220386965, Dec. 8, 2022.

GGGG. U.S. Utility patent application Ser. No. 17/339,153, entitled “Method and System for the Safety, Analysis, and Supervision of Insulin Pump Action and Other Modes of Insulin Delivery in Diabetes”, filed Jun. 4, 2021; Publication No. US 2021-0313036 A1, Oct. 7, 2021.

HHHH. U.S. Utility patent application Ser. No. 13/634,040, entitled “Method and System for the Safety, Analysis, and Supervision of Insulin Pump Action and Other Modes of Insulin Delivery in Diabetes”, filed Sep. 11, 2012; U.S. Pat. No. 11,069,434, issued Jul. 20, 2021; Publication No. 2013/0116649, May 9, 2013.

IIII. International Patent Application Serial No. PCT/US2011/028163, entitled “Method and System for the Safety, Analysis, and Supervision of Insulin Pump Action and Other Modes of Insulin Delivery in Diabetes”, filed Mar. 11, 2011; Publication No. WO 2011/112974, Sep. 15, 2011.

JJJJ. International Patent Application Serial No. PCT/US2013/042745, entitled “INSULIN-PRAMLINTIDE COMPOSITIONS AND METHODS FOR MAKING AND USING THEM”, filed May 24, 2013; Publication No. WO 2013/177565, Nov. 28, 2013.

KKKK. U.S. Utility patent application Ser. No. 13/637,359, entitled “METHOD, SYSTEM, AND COMPUTER PROGRAM PRODUCT FOR IMPROVING THE ACCURACY OF GLUCOSE SENSORS USING INSULIN DELIVERY OBSERVATION IN DIABETES”, filed Sep. 25, 2012; U.S. Pat. No. 9,398,869, issued Jul. 26, 2016; Publication No. 2013/0079613, Mar. 28, 2013.

LLLL. International Patent Application Serial No. PCT/US2011/029793, entitled “METHOD, SYSTEM, AND COMPUTER PROGRAM PRODUCT FOR IMPROVING THE ACCURACY OF GLUCOSE SENSORS USING INSULIN DELIVERY OBSERVATION IN DIABETES”, filed Mar. 24, 2011; Publication No. WO 2011/119832, Sep. 29, 2011.

MMMM. U.S. Utility patent application Ser. No. 17/590,659, entitled “System, Method, and Computer Simulation Environment for In Silico Trials in Pre-Diabetes and Type 2 Diabetes”, filed Feb. 1, 2022; Publication No. US 2022-0230762 A1, Jul. 21, 2022.

NNNN. U.S. Utility patent application Ser. No. 13/380,839, entitled “System, Method, and Computer Simulation Environment for In Silico Trials in Pre-Diabetes and Type 2 Diabetes”, filed Dec. 25, 2011; U.S. Pat. No. 11,238,990, issued Feb. 1, 2022; Publication No. 2012/0130698, May 24, 2012.

OOOO. International Patent Application Serial No. PCT/US2010/040097, entitled “System, Method, and Computer Simulation Environment for In Silico Trials in Prediabetes and Type 2 Diabetes”, filed Jun. 25, 2010; Publication No. WO 2010/151834, Dec. 29, 2010.

PPPP. U.S. Utility patent application Ser. No. 18/278,549, entitled “METHOD AND SYSTEM FOR QUANTITATIVE PHYSIOLOGICAL ASSESSMENT AND PREDICTION OF CLINICAL SUBTYPES OF GLUCOSE METABOLISM DISORDERS”, filed Aug. 23, 2023.

QQQQ. International Patent Application Serial No. PCT/US2022/017489, entitled “METHOD AND SYSTEM FOR QUANTITATIVE PHYSIOLOGICAL ASSESSMENT AND PREDICTION OF CLINICAL SUBTYPES OF GLUCOSE METABOLISM DISORDERS”, filed Feb. 23, 2022; Publication No. WO 2022/182736, Sep. 1, 2022.

RRRR. U.S. Utility patent application Ser. No. 12/664,444, entitled “Method, System and Computer Simulation Environment for Testing of Monitoring and Control Strategies in Diabetes”, filed Dec. 14, 2009; U.S. Pat. No. 10,546,659, issued Jan. 28, 2020; Publication No. 2010/0179768, Jul. 15, 2010.

SSSS. International Patent Application Serial No. PCT/US2008/067725, entitled “Method, System and Computer Simulation Environment for Testing of Monitoring and Control Strategies in Diabetes”, filed Jun. 20, 2008; Publication No. WO 2008/157781, Dec. 24, 2008.

TTTT. U.S. Utility patent application Ser. No. 14/902,731, entitled “SIMULATION OF ENDOGENOUS AND EXOGENOUS GLUCOSE/INSULIN/GLUCAGON INTERPLAY IN TYPE 1 DIABETIC PATIENTS”, filed Jan. 4, 2016; U.S. Pat. No. 10,169,544, issued Jan. 1, 2019; Publication No. US-2016-0171183-A1, Jun. 16, 2016.

UUUU. International Patent Application Serial No. PCT/US2014/045393, entitled “SIMULATION OF ENDOGENOUS AND EXOGENOUS GLUCOSE/INSULIN/GLUCAGON INTERPLAY IN TYPE 1 DIABETIC PATIENTS”, filed Jul. 3, 2014; Publication No. WO2015003124, Jan. 8, 2015.

VVVV. U.S. Utility patent application Ser. No. 18/278,485, entitled “METHOD AND SYSTEM FOR MAPPING INDIVIDUALIZED METABOLIC PHENOTYPE TO A DATABASE IMAGE FOR OPTIMIZING CONTROL OF CHRONIC METABOLIC CONDITIONS”, filed Aug. 23, 2023.

WWWW. International Patent Application Serial No. PCT/US2022/017449, entitled “METHOD AND SYSTEM FOR MAPPING INDIVIDUALIZED METABOLIC PHENOTYPE TO A DATABASE IMAGE FOR OPTIMIZING CONTROL OF CHRONIC METABOLIC CONDITIONS”, filed Feb. 23, 2022; Publication No. WO2022182709, Sep. 1, 2022.

XXXX. U.S. Utility patent application Ser. No. 13/131,467, entitled “Method, System, and Computer Program Product for Tracking of Blood Glucose Variability in Diabetes”, filed May 26, 2011; U.S. Pat. No. 9,317,657, issued Apr. 19, 2016; Publication No. 2011/0264378, Oct. 27, 2011.

YYYY. International Patent Application Serial No. PCT/US2009/065725, entitled “Method, System, and Computer Program Product for Tracking of Blood Glucose Variability in Diabetes”, filed Nov. 24, 2009; Publication No. WO 2010/062898, Jun. 3, 2010.

ZZZZ. U.S. Utility patent application Ser. No. 10/069,674, entitled “Method and Apparatus for Predicting the Risk of Hypoglycemia”, filed Feb. 22, 2002; U.S. Pat. No. 6,923,763, issued Aug. 2, 2005.

AAAAA. International Patent Application Serial No. US00/22886, entitled “METHOD AND APPARATUS FOR PREDICTING THE RISK OF HYPOGLYCEMIA”, filed Aug. 21, 2000; Publication No. WO01/13786, Mar. 1, 2001.

BBBBB. International Patent Application Serial No. PCT/US2002/005676, entitled “METHOD AND APPARATUS FOR THE EARLY DIAGNOSIS OF SUBACUTE, POTENTIALLY CATASTROPHIC ILLNESS”, filed Feb. 27, 2002; Publication No. WO02/67776, Sep. 6, 2002.

CCCCC. U.S. Utility patent application Ser. No. 09/793,653, entitled “METHOD AND APPARATUS FOR THE EARLY DIAGNOSIS OF SUBACUTE, POTENTIALLY CATASTROPHIC ILLNESS”, filed Feb. 27, 2001; U.S. Pat. No. 6,804,551, issued Oct. 12, 2004; Publication No. 2002/0052557, May 2, 2002.

DDDDD. U.S. Utility patent application Ser. No. 14/241,383, entitled “Method, System and Computer Readable Medium for Adaptive Advisory Control of Diabetes”, filed Feb. 26, 2014; U.S. Pat. No. 11,024,429, issued Jun. 1, 2021; Publication No. 2015-0190098, Jul. 9, 2015.

EEEEE. International Patent Application Serial No. PCT/US2012/052422, entitled “Method, System and Computer Readable Medium for Adaptive Advisory Control of Diabetes”, filed Aug. 26, 2012; Publication No. WO 2013/032965, Mar. 7, 2013.

FFFFF. U.S. Utility patent application Ser. No. 14/419,375, entitled “COMPUTER SIMULATION FOR TESTING AND MONITORING OF TREATMENT STRATEGIES FOR STRESS HYPERGLYCEMIA”, filed Feb. 3, 2015; U.S. Pat. No. 10,438,700, issued Oct. 8, 2019; Publication No. 2015-0193589, Jul. 9, 2015.

GGGGG. International Patent Application Serial No. PCT/US2013/053664, entitled “COMPUTER SIMULATION FOR TESTING AND MONITORING OF TREATMENT STRATEGIES FOR STRESS HYPERGLYCEMIA”, filed Aug. 5, 2013; Publication No. WO 2014/022864, Feb. 6, 2014.

HHHHH. U.S. Utility patent application Ser. No. 18/390,081, entitled “SYSTEM COORDINATOR AND MODULAR ARCHITECTURE FOR OPEN-LOOP AND CLOSED-LOOP CONTROL OF DIABETES”, filed Dec. 20, 2023.

IIIII. U.S. Utility patent application Ser. No. 18/333,955, entitled “System Coordinator and Modular Architecture for Open-Loop and Closed-Loop Control of Diabetes”, filed Jun. 13, 2023; Publication No. US 2023-0368885 A1, Nov. 16, 2023.

JJJJJ. U.S. Utility patent application Ser. No. 18/303,962, entitled “System Coordinator and Modular Architecture for Open-Loop and Closed-Loop Control of Diabetes”, filed Apr. 20, 2023; Publication No. US2023-0260617A1, Aug. 17, 2023.

KKKKK. U.S. Utility patent application Ser. No. 16/546,335, entitled “System Coordinator and Modular Architecture for Open-Loop and Closed-Loop Control of Diabetes”, filed Aug. 21, 2019; Publication No. US-2019-0374137-A1, Dec. 12, 2019.

LLLLL. U.S. Utility patent application Ser. No. 13/322,943, entitled “System Coordinator and Modular Architecture for Open-Loop and Closed-Loop Control of Diabetes”, filed Nov. 29, 2011; U.S. Pat. No. 10,420,489, issued Sep. 24, 2019; Publication No. 2012/0078067, Mar. 29, 2012.

MMMMM. International Patent Application Serial No. PCT/US2010/036629, entitled “System Coordinator and Modular Architecture for Open-Loop and Closed-Loop Control of Diabetes”, filed May 28, 2010; Publication No. WO 2010/138848, Dec. 2, 2010.

NNNNN. U.S. Utility patent application Ser. No. 17/776,056, entitled “SYSTEM, METHOD AND COMPUTER READABLE MEDIUM FOR COMPRESSING CONTINUOUS GLUCOSE MONITOR DATA”, filed May 11, 2022; Publication No. 20220392632, Dec. 8, 2022.

OOOOO. International Patent Application Serial No. PCT/US2020/060366, entitled “SYSTEM, METHOD AND COMPUTER READABLE MEDIUM FOR COMPRESSING CONTINUOUS GLUCOSE MONITOR DATA”, filed Nov. 13, 2020; Publication No. WO 2021/097178, May 20, 2021.

In summary, while the present invention has been described with respect to specific embodiments, many modifications, variations, alterations, substitutions, and equivalents will be apparent to those skilled in the art. The present invention is not to be limited in scope by the specific embodiment described herein. Indeed, various modifications of the present invention, in addition to those described herein, will be apparent to those of skill in the art from the foregoing description and accompanying drawings. Accordingly, the invention is to be considered as limited only by the spirit and scope of the following claims including all modifications and equivalents.

Still other embodiments will become readily apparent to those skilled in this art from reading the above-recited detailed description and drawings of certain exemplary embodiments. It should be understood that numerous variations, modifications, and additional embodiments are possible, and accordingly, all such variations, modifications, and embodiments are to be regarded as being within the spirit and scope of this application. For example, regardless of the content of any portion (e.g., title, field, background, summary, abstract, drawing figure, etc.) of this application, unless clearly specified to the contrary, there is no requirement for the inclusion in any claim herein or of any application claiming priority hereto of any particular described or illustrated activity or element, any particular sequence of such activities, or any particular interrelationship of such elements. Moreover, any activity can be repeated, any activity can be performed by multiple entities, and/or any element can be duplicated. Further, any activity or element can be excluded, the sequence of activities can vary, and/or the interrelationship of elements can vary. Unless clearly specified to the contrary, there is no requirement for any particular described or illustrated activity or element, any particular sequence or such activities, any particular size, speed, material, dimension or frequency, or any particular interrelationship of such elements. Accordingly, the descriptions and drawings are to be regarded as illustrative in nature, and not as restrictive. Moreover, when any number or range is described herein, unless clearly stated otherwise, that number or range is approximate. When any range is described herein, unless clearly stated otherwise, that range includes all values therein and all sub ranges therein. Any information in any material (e.g., a United States/foreign patent, United States/foreign patent application, book, article, etc.) that has been incorporated by reference herein, is only incorporated by reference to the extent that no conflict exists between such information and the other statements and drawings set forth herein. In the event of such conflict, including a conflict that would render invalid any claim herein or seeking priority hereto, then any such conflicting information in such incorporated by reference material is specifically not incorporated by reference herein.

What is claimed is:

1. A processor-based method for improving human-machine co-regulation of metabolic disorders, the method comprising:
receiving a user's historical data, the user's historical data including the user's glucose monitoring records and insulin therapy profile;
determining a plurality of actionable information and risk assessments to assist the user's behavioral intervention based on user's glucose monitoring records;
processing said historical data to estimate a personalized model of the user's glucose metabolism;
determining insulin-dosing parameters based on said personalized model of the user's glucose metabolism; and
computing a simulation of effects from hypothetical changes to the user's treatment of behavior based on said personalized model of the user's glucose metabolism.

2. The method of claim 1, wherein the plurality of actionable information and risk assessments include one or more of the following:
the user's time spent in different glucose ranges;
a glucose variability index which tracks daily glucose variation;
the user's glucose median and percentiles across a period of time;
the user's time spent in different glucose ranges;
an estimate mean glucose (EMG); and
the user's hypoglycemia and hyperglycemia risk indices.

3. The method of claim 2, wherein, for a specified duration, the estimate mean glucose is computed from received historical data, wherein said historical data includes at least 70% of continuous glucose monitoring from a closed time period prior to the calculation.

4. The method of claim 3, wherein the formula used to compute the estimate mean glucose is:
$$EMG ( \% ) = 3.31 + 0.02392 \times [ mean glucose in mg / dl ] .$$

5. The method of claim 2, wherein, if less than 70% of continuous glucose monitoring data from a preceding specified duration is available, the trend of the estimate mean glucose change over the previous seven days is estimated through linear regression and mapped into a specific “arrow slope,” wherein said arrow slope represents the estimate mean glucose rate of change.

6. The method of claim 5, wherein the estimate mean glucose rate of change (emgROC) is based on the lookup table:

| emgROC | Arrow Slope |
|---|---|
| <=a, | Rapid decrease |
| where a < −1.0 mg/dL/min | (code: −2) |
| >a AND <=b, | Moderate decrease |
| where a < −1.0 mg/dL/min | (code: −1) |
| where b is between −1.0 and −0.25 mg/dL/min | |
| >b AND <=c, | No relevant change |
| where b is between −1.0 and −0.25 mg/dL/min | (code: 0) |
| where c is between 0.25 and 1.0 mg/dL/min | |
| >c AND <=d, | Moderate increase |
| where c is between 0.25 and 1.0 mg/dL/min | (code: +1) |
| where d > 1 mg/dL/min | |
| >=d, | Rapid increase |
| where d > 1.0 mg/dL/min. | (code: +2) |

7. The method of claim 2, wherein the formulas used to compute said hypoglycemia risk index (low blood glucose index (LBGI)) and said hyperglycemia risk index (high blood glucose index (HBGI)) are:
$$LBGI = \frac{\sum {r}_{l} ( {x}_{i} )}{n}$$
where rₗ(xᵢ)=22.77f(xᵢ)² if f(xᵢ)²≤0, and 0 otherwise; and
$$HBGI = \frac{\sum {r}_{h} ( {x}_{i} )}{n}$$
where rₕ(xᵢ)=22.77 f(xᵢ)² if f(xᵢ)>0, and 0 otherwise, and where
f(xᵢ)=(ln(xᵢ)^{1.804}-5.381 3for blood glucose readings x₁, . . . , xₙ measured in mg/dL.

8. The method of claim 1, wherein said historical data further includes the user's meal history and/or controller-specific records.

9. The method of claim 1, further comprising: executing a logistic regression-based detection algorithm to reconstruct and amend a meal record of the user.

10. The method of claim 1, further comprising: identifying, for a specified duration of said historical data, said personalized model of the user's glucose metabolism in the presence of meal ingestion and subcutaneous insulin administration.

11. The method of claim 1, further comprising: estimating an additive net-effect signal through model inversion by deconvolution based on said personalized model.

12. The method of claim 9, wherein the personalized model of the user's glucose metabolism comprises:
identifying a personalized model of the user's glucose metabolism in the presence of meal ingestion and subcutaneous insulin administration; and
estimating an additive net-effect signal through model inversion by deconvolution.

13. The method of claim 11, further comprising: recording processed data, model parameters, and net-effect signal in a system database.

14. The method of claim 10, further comprising: periodically replaying glucose traces represented by the personalized model of the user's glucose metabolism in a closed-loop fashion with an automated insulin delivery system.

15. The method of claim 14, further comprising: associating said replayed glucose traces with a root mean square error.

16. The method of claim 15, wherein said glucose traces with a root mean square error above a predefined threshold are excluded from the dataset used to determine insulin-dosing parameters.

17. The method of claim 1, wherein said insulin-dosing parameters are determined by solving an optimization problem that minimizes the weighted sum of the risks of hypoglycemia (LBGI) and hyperglycemia (HBGI) and the number of hypoglycemic treatments.

18. The method of claim 1, wherein said insulin-dosing parameters are determined by solving the optimization problem:
$${min}_{\{ {α}_{1} , … , {α}_{6} \} \{ {β}_{1} , … , {β}_{6} \}} J = f ( {BR}^{′} , {CR}^{′} , {CF}^{′} )$$
$$subject to$$
$${α}_{i} ∈ [ - 1 , 1 ] ∀ i = 1 , … , 6$$
$${β}_{i} ∈ [ - 1 , 1 ] ∀ i = 1 , … , 6$$
with α^{v}={α₁, . . . , α₆} and β^{v}={β₁, . . . , β₆] the vector of modulation factors for CR-CF and BR in segments with predefined lengths, and BR′, CR′, and CF′, the modulated profiles through the day for BR and CR-CF computed as a function of α^{v} and β^{v}:
$${BR}^{′} = {BR}_{nom}^{v} \cdot ( 1 + P * \frac{{β}_{ext}^{v}}{100} )$$
$${CR}^{′} = {CR}_{nom}^{v} \cdot ( 1 + P * \frac{{α}_{ext}^{v}}{100} )$$
$${CF}^{′} = {CF}_{nom}^{v} \cdot ( 1 + P * \frac{{α}_{ext}^{v}}{100} )$$
with
$${BR}_{nom}^{v} , {CR}_{nom}^{v} and {CF}_{nom}^{v}$$
the current profiles through the day for BR, CR, and CF in five-minute intervals, and
$${α}_{ext}^{v}$$
and βₑₓₜ^{v} the extended version of α^{v} and β^{v} through the day in five-minute intervals; and
where the cost function (J) is:
$$J = {{\sum }_{j = 1}}^{{N}_{days}} 2 \cdot {LBGI}_{j} + {HBGI}_{j} + {N}_{j , HT}$$
with N_{days} the number of days considered for the optimization (≤14 days), N_{HT} the number of hypoglycemic treatments per day, and LBGI; and HBGI; the hypoglycemia and hyperglycemia risk indices per day.

19. The method of claim 1, wherein the historical data further includes a finite time range and basal and bolus modulators.

20. The method of claim 1, wherein the said simulation is based on custom modulation factors.

21. The method of claim 20, wherein said custom modulation factors are determined by parsing received historical data into finite time ranges with associated basal and bolus modulators based on said personalized model of the user's glucose metabolism.

22. The method of claim 20, wherein said custom modulation factors are defined as Pfactors.

23. The method of claim 22, wherein the said simulation is based on combining optimal modulation factors with said Pfactors to define final modulation profiles for at least one of: basal rate, carbohydrate ratio, and correction factor.

24. The method of claim 1, wherein said simulation features changes to at least one of: basal rate profile, carbohydrate ratio profile, correction factor profiles, or user-specific glucose charts.

25. The method of claim 1, wherein said treatment includes changes to basal and bolus settings.

26. The method of claim 1, further comprising presenting the time spent in different glucose ranges via commonly accepted glucose charts.

27. The method of claim 1, further comprising one or more of the following:
recording said plurality of actionable information and risk assessments to a database;
recording said plurality of actionable information and risk assessments to a database accessible by the user;
presenting said plurality of actionable information and risk assessments to the user via a display system;
presenting said plurality of actionable information and risk assessments to the user via a website or web application;
recording said insulin-dosing parameters based on the personalized model of the user's glucose metabolism to a database,
recording said insulin-dosing parameters based on the personalized model of the user's glucose metabolism to a database accessible by the user;
presenting said insulin-dosing parameters based on the personalized model of the user's glucose metabolism via a display system; and
presenting said insulin-dosing parameters based on the personalized model of the user's glucose metabolism via a website or web application.

28. The method of claim 1, wherein at least part of the historical data is obtained from one or more of the following:
an insulin pump device, continuous glucose monitoring device, a manual insulin injection device, a meal-tracking device, and an activity-tracking device.

29. The method of claim 1, wherein the insulin-dosing parameters are recommended to the user or automatically implemented by a closed-loop control or continuous subcutaneous insulin delivery system.

30. A system for improving human-machine co-regulation of metabolic disorders, the system comprising:
a computer processor;
a memory configured to store instructions that are executable by said computer processor, wherein said computer processor is configured to execute instructions for:
receiving a user's historical data, the user's historical data including the user's glucose monitoring records and insulin therapy profile;
determining a plurality of actionable information and risk assessments to assist the user's behavioral intervention based on user's glucose monitoring records, processing said historical data to estimate a personalized model of the user's glucose metabolism;
determining insulin-dosing parameters based on said personalized model of the user's glucose metabolism; and
computing a simulation of effects from hypothetical changes or non-changes to the user's treatment or behavior based on said personalized model of the user's glucose metabolism.

31. The system of claim 30, wherein the plurality of actionable information and risk assessments include one or more of the following:
the user's time spent in different glucose ranges;
a glucose variability index which tracks daily glucose variation;
the user's glucose median and percentiles across a period of time;
the user's time spent in different glucose ranges;
an estimate mean glucose (EMG); and
the user's hypoglycemia and hyperglycemia risk indices.

32. The system of claim 31, wherein, for a specified duration, the estimate mean glucose is computed from received historical data, wherein said historical data includes at least 70% of continuous glucose monitoring from a closed time period prior to the calculation.

33. The system of claim 32, wherein the formula used to compute the estimate mean glucose is:
$$EMG ( \% ) = 3.31 + 0.02392 \times [ mean glucose in mg / dl ] .$$

34. The system of claim 31, wherein, if less than 70% of continuous glucose monitoring data from the preceding specified duration is available, the trend of the estimate mean glucose change over the previous seven days is estimated through linear regression and mapped into a specific “arrow slope,” wherein said arrow slope represents the estimate mean glucose rate of change.

35. The system of claim 34, wherein the estimate mean glucose rate of change (emgROC) is based on the lookup table:

| emgROC | Arrow Slope |
|---|---|
| <=a, | Rapid decrease |
| where a < −1.0 mg/dL/min | (code: −2) |
| >a AND <=b, | Moderate decrease |
| where a < −1.0 mg/dL/min | (code: −1) |
| where b is between −1.0 and −0.25 mg/dL/min | |
| >b AND <=c, | No relevant change |
| where b is between −1.0 and −0.25 mg/dL/min | (code: 0) |
| where c is between 0.25 and 1.0 mg/dL/min | |
| >c AND <=d, | Moderate increase |
| where c is between 0.25 and 1.0 mg/dL/min | (code: +1) |
| where d > 1 mg/dL/min | |
| >=d, | Rapid increase |
| where d > 1.0 mg/dL/min. | (code: +2) |

36. The system of claim 31, wherein the formulas used to compute said hypoglycemia risk index (low blood glucose index (LBGI)) and said hyperglycemia risk index (high blood glucose index (HBGI)) are:
$$LBGI = \frac{\sum {r}_{l} ( {x}_{i} )}{n}$$
where rₗ(xᵢ)=22.77f(xᵢ)² if f(xᵢ)≤0, and 0 otherwise; and
$$HBGI = \frac{\sum {r}_{h} ( {x}_{i} )}{n}$$
where rₕ(xᵢ)=22.77f(xᵢ)² if f(xᵢ)>0, and 0 otherwise, and where
f(xᵢ)=(ln(xᵢ)^{1.804}-5.381 3for blood glucose readings x₁, . . . , xₙ measured in mg/dL.

37. The system of claim 30, wherein said historical data further includes the user's meal history and/or controller-specific records.

38. The system of claim 30, wherein said computer processor is further configured to execute instructions for:
computing a logistic regression-based detection algorithm to reconstruct and amend a meal record of the user.

39. The system of claim 30, wherein said computer processor is further configured to execute instructions for:
identifying, for a specified duration of said historical data, said personalized model of the user's glucose metabolism in the presence of meal ingestion and subcutaneous insulin administration.

40. The system of claim 30, wherein said computer processor is further configured to execute instructions for: estimating an additive net-effect signal through model inversion by deconvolution based on said personalized model.

41. The system of claim 38, wherein the personalized model of the user's glucose metabolism comprises:
identifying a personalized model of the user's glucose metabolism in the presence of meal ingestion and subcutaneous insulin administration; and
estimating an additive net-effect signal through model inversion by deconvolution.

42. The system of claim 40, wherein said computer processor is further configured to execute instructions for: recording processed data, model parameters, and net-effect signal in a system database.

43. The system of claim 39, further configured to execute instructions for:
periodically replaying glucose traces represented by the personalized model of the user's glucose metabolism in a closed-loop fashion with an automated insulin delivery system.

44. The system of claim 43, wherein said computer processor is further configured to execute instructions for: associating said replayed glucose traces with a root mean square error.

45. The system of claim 44, wherein said glucose traces with a root mean square error above a predefined threshold are excluded from the dataset used to determine insulin-dosing parameters.

46. The system of claim 30, wherein said insulin-dosing parameters are determined by solving an optimization problem that minimizes the weighted sum of the risks of hypoglycemia (LBGI) and hyperglycemia (HBGI) and the number of hypoglycemic treatments.

47. The system of claim 30, wherein said insulin-dosing parameters are determined by solving the optimization problem:
$${min}_{\{ {α}_{1} , … , {α}_{6} \} \{ {β}_{1} , … , {β}_{6} \}} J = f ( {BR}^{′} , {CR}^{′} , {CF}^{′} )$$
$$subject to$$
$${α}_{i} ∈ [ - 1 , 1 ] ∀ i = 1 , … , 6$$
$${β}_{i} ∈ [ - 1 , 1 ] ∀ i = 1 , … , 6$$
with α^{v}={α₁, . . . , α₆} and β^{v}={β₁, . . . , β₆} the vector of modulation factors
for CR-CF and BR in segments with predefined lengths, and BR′, CR′, and
CF′, the modulated profiles through the day for BR and CR-CF computed as a function of α^{v} and β^{v}:
$${BR}^{′} = {BR}_{nom}^{v} \cdot ( 1 + P * \frac{{β}_{ext}^{v}}{100} )$$
$${CR}^{′} = {CR}_{nom}^{v} \cdot ( 1 + P * \frac{{α}_{ext}^{v}}{100} )$$
$${CF}^{′} = {CF}_{nom}^{v} \cdot ( 1 + P * \frac{{α}_{ext}^{v}}{100} )$$
with
$${BR}_{nom}^{v} , {CR}_{nom}^{v} and {CF}_{nom}^{v}$$
the current profiles through the day for BR, CR, and CF in five-minute intervals, and
$${α}_{ext}^{v}$$
and βₑₓₜ^{v} the extended version of α^{v} and β^{v} through the day in five-minute intervals; and
where the cost function (J) is:
$$J = {\sum }_{j = 1}^{{N}_{days}} 2 \cdot {LBGI}_{j} + {HBGI}_{j} + {N}_{j , HT}$$
with N_{days} the number of days considered for the optimization (≤14 days), Nur the number of hypoglycemic treatments per day, and LBGI, and HBGI, the hypoglycemia and hyperglycemia indices per day.

48. The system of claim 30, wherein the historical data further includes a finite time range and basal and bolus modulators.

49. The system of claim 30, wherein the said simulation is based on custom modulation factors.

50. The system of claim 49, wherein said custom modulation factors are determined by parsing received historical data into finite time ranges with associated basal and bolus modulators based on said personalized model of the user's glucose metabolism.

51. The system of claim 49, wherein said custom modulation factors are defined as Pfactors.

52. The system of claim 51, wherein the said simulation is based on combining optimal modulation factors with said Pfactors to define final modulation profiles for at least one of: basal rate, carbohydrate ratio, and correction factor.

53. The system of claim 30, wherein said simulation features changes or non-changes to at least one of: basal rate profile, carbohydrate ratio profile, correction factor profiles, or user-specific glucose charts.

54. The system of claim 30, wherein said treatment includes changes or non-changes to basal and bolus settings.

55. The system of claim 30, further comprising presenting the time spent in different glucose ranges via commonly accepted glucose charts.

56. The system of claim 30, wherein said computer processor is further configured to execute instructions for one or more of the following:
recording said plurality of actionable information and risk assessments to a database;
recording said plurality of actionable information and risk assessments to a database accessible by the user;
presenting said plurality of actionable information and risk assessments to the patient via a display system;
presenting said plurality of actionable information and risk assessments to the user via a website or web application;
recording said insulin-dosing parameters based on the personalized model of the user's glucose metabolism to a database;
recording said insulin-dosing parameters based on the personalized model of the user's glucose metabolism to a database accessible by the user;
presenting said insulin-dosing parameters based on the personalized model of the user's glucose metabolism via a display system; and
presenting said insulin-dosing parameters based on the personalized model of the user's glucose metabolism via a website or web application.

57. The system of claim 30, wherein said system further comprises one or more of the following:
a portal configured to manage user accounts;
a database used to store user data and programmed instructions for system operation; and
a user interface capable of displaying content and sending notifications to the user.

58. The system of claim 57, wherein said portal is a web-based portal.

59. The system of claim 57, wherein:
said system is configured to communicate with a third-party system.

60. The system of claim 59, wherein:
said third party system is configured to communicate with a server coupled with a network; and
wherein said third-party system is configured to upload the user's historical data to the processor by way of the server.

61. The system of claim 59, wherein said third-party system is connected to the Internet that is capable of connecting to and uploading data from at least one of: an insulin monitoring device, an insulin delivery device, or a glucose monitoring device.

62. The system of claim 59, wherein said third party system includes one or more of the following: mobile app, tablet, computer, mobile device, personal electronic device, personal digital assistant (PDA), or smartwatch.

63. The system of claim 30, wherein at least part of the historical data is obtained or uploaded from one or more of the following:
an insulin pump device, continuous glucose monitoring device, a manual insulin injection device, a meal-tracking device, and an activity-tracking device.

64. The system of claim 30, wherein the insulin-dosing parameters are recommended to the user or automatically implemented by a closed-loop control or continuous subcutaneous insulin delivery system.

65. A non-transitory computer readable medium having program instructions for use in human-machine co-regulation of metabolic disorders, wherein execution of the program instructions by one or more processors of a computer system causes the one or more processors to:
receive a user's historical data, the user's historical data including the user's glucose monitoring records and insulin therapy profile;
determine a plurality of actionable information and risk assessments to assist the user's behavioral intervention based on user's glucose monitoring records;
process said historical data to estimate a personalized model of the user's glucose metabolism;
determine insulin-dosing parameters based on said personalized model of the user's glucose metabolism; and
compute a simulation of effects from hypothetical changes to the user's treatment or behavior based on said personalized model of the user's glucose metabolism.

66. The non-transitory computer readable medium of claim 65, wherein execution of the program instructions by one or more processors of the computer system further causes the one or more processors to:
communicate with a third party system.

67. The non-transitory computer readable medium of claim 66, wherein, said third party system is configured to communicate with a server coupled to a network, and
said third-party system is configured to upload the user's historical data to the one or more processors by way of the server.

68. The non-transitory computer readable medium of claim 66, wherein execution of the program instructions by one or more processors of the computer system further causes the one or more processors to: display content and send notifications to the user via a user interface.

69. The non-transitory computer readable medium of claim 66, wherein said third-party system connected to the Internet that is capable of connecting to and uploading data from at least one of:
an insulin monitoring device, an insulin delivery device, or a glucose monitoring device.

70. The non-transitory computer readable medium of claim 66, wherein said third party system includes one or more of the following: mobile app, tablet, computer, mobile device, personal electronic device, personal digital assistant (PDA), or smartwatch.

71. The non-transitory computer readable medium of claim 65, wherein the plurality of actionable information and risk assessments include one or more of the following:
the user's time spent in different glucose ranges;
a glucose variability index which tracks daily glucose variation;
the user's glucose median and percentiles across a period of time;
the user's time spent in different glucose ranges;
an estimate mean glucose (EMG); and
the user's hypoglycemia and hyperglycemia risk indices.

72. The system of claim 71, wherein, for a specified duration, the estimate mean glucose is computed from received historical data, wherein said historical data includes at least 70% of continuous glucose monitoring from a closed time period prior to the calculation.

73. The non-transitory computer readable medium of claim 72, wherein the formula used to compute the estimate mean glucose is:
EMG(%)=3.31+0.02392×[mean glucose in mg/dl].

74. The non-transitory computer readable medium of claim 71, wherein, if less than 70% of continuous glucose monitoring data from a preceding specified duration is available, the trend of the estimate mean glucose change over the previous seven days is estimated through linear regression and mapped into a specific “arrow slope,” wherein said arrow slope represents the estimate mean glucose rate of change.

75. The non-transitory computer readable medium of claim 74, wherein the estimate mean glucose rate of change (emgROC) is based on the lookup table:

| emgROC | Arrow Slope |
|---|---|
| <=a, | Rapid decrease |
| where a < −1.0 mg/dL/min | (code: −2) |
| >a AND <=b, | Moderate decrease |
| where a < −1.0 mg/dL/min | (code: −1) |
| where b is between −1.0 and −0.25 mg/dL/min | |
| >b AND <=c, | No relevant change |
| where b is between −1.0 and −0.25 mg/dL/min | (code: 0) |
| where c is between 0.25 and 1.0 mg/dL/min | |
| >c AND <=d, | Moderate increase |
| where c is between 0.25 and 1.0 mg/dL/min | (code: +1) |
| where d > 1 mg/dL/min | |
| >=d, | Rapid increase |
| where d > 1.0 mg/dL/min. | (code: +2) |

76. The non-transitory computer readable medium of claim 71, wherein the formulas used to compute said hypoglycemia risk index (low blood glucose index (LBGI)) and said hyperglycemia risk index (high blood glucose index (HBGI)) are:
$$LBGI = \frac{\sum {r}_{l} ( {x}_{i} )}{n}$$
where rₗ(xᵢ)=22.77f(xᵢ)² if f(xᵢ)≤0, and 0 otherwise; and
$$HBGI = \frac{\sum {r}_{h} ( {x}_{i} )}{n}$$
where rₕ(x)=22.77f(xᵢ)² if f(xᵢ)>0, and 0 otherwise, and where
f(xᵢ)=(ln(xᵢ)^{1.804}-5.381 3for blood glucose readings x₁, . . . , xₙ measured in mg/dL.

77. The non-transitory computer readable medium of claim 65, wherein said historical data further includes the user's meal history and/or controller-specific records.

78. The non-transitory computer readable medium of claim 65, wherein said computer processor is further configured to execute instructions for:
computing a logistic regression-based detection algorithm to reconstruct and amend a meal record of the user.

79. The non-transitory computer readable medium of claim 65, wherein execution of the program instructions by said one or more computer processors cause the one or more processors to:
identify, for a specified duration of said historical data, said personalized model of the user's glucose metabolism in the presence of meal ingestion and subcutaneous insulin administration.

80. The non-transitory computer readable medium of claim 65, wherein execution of the program instructions by said one or more computer processors cause the one or more processors to:
estimate an additive net-effect signal through model inversion by deconvolution based on said personalized model.

81. The non-transitory computer readable medium of claim 78, wherein the personalized model of the user's glucose metabolism comprises:
identifying a personalized model of the user's glucose metabolism in the presence of meal ingestion and subcutaneous insulin administration; and
estimating an additive net-effect signal through model inversion by deconvolution.

82. The non-transitory computer readable medium of claim 80, wherein execution of the program instructions by said one or more computer processors cause the one or more processors to:
record processed data, model parameters, and net-effect signal in a system database.

83. The non-transitory computer readable medium of claim 79, wherein execution of the program instructions by said one or more computer processors cause the one or more processors to:
periodically replay glucose traces represented by the personalized model of the user's glucose metabolism in a closed-loop fashion with an automated insulin delivery system.

84. The non-transitory computer readable medium of claim 83, wherein execution of the program instructions by said one or more computer processors cause the one or more processors to:
associate said replayed glucose traces with a root mean square error.

85. The non-transitory computer readable medium of claim 84, wherein said glucose traces with a root mean square error above a predefined threshold are excluded from the dataset used to determine insulin-dosing parameters.

86. The non-transitory computer readable medium of claim 65, wherein said insulin-dosing parameters are determined by solving an optimization problem that minimizes the weighted sum of the risks of hypoglycemia (LBGI) and hyperglycemia (HBGI) and the number of hypoglycemic treatments.

87. The non-transitory computer readable medium of claim 65, wherein said insulin-dosing parameters are determined by solving the optimization problem:
$${min}_{\{ {α}_{1} , … , {α}_{6} \} \{ {β}_{1} , … , {β}_{6} \}} J = f ( {BR}^{′} , {CR}^{′} , {CF}^{′} )$$
$$subject to$$
$${α}_{i} ∈ [ - 1 , 1 ] ∀ i = 1 , … , 6$$
$${β}_{i} ∈ [ - 1 , 1 ] ∀ i = 1 , … , 6$$
with α^{v}={β₁, . . . , α₆} and β^{v}={β₁, . . . , β₆} the vector of modulation factors for CR-CF and BR in segments with predefined lengths, and BR′, CR′, and CF′, the modulated profiles through the day for BR and CR-CF computed as a function of α^{v} and β^{v}:
$${BR}^{′} = {BR}_{nom}^{v} \cdot ( 1 + P * \frac{{β}_{ext}^{v}}{100} )$$
$${CR}^{′} = {CR}_{nom}^{v} \cdot ( 1 + P * \frac{{α}_{ext}^{v}}{100} )$$
$${CF}^{′} = {CF}_{nom}^{v} \cdot ( 1 + P * \frac{{α}_{ext}^{v}}{100} )$$
with
$${BR}_{nom}^{v} , {CR}_{nom}^{v} and {CF}_{nom}^{v}$$
the current profiles through the day for BR, CR, and CF in five-minute intervals, and
$${α}_{ext}^{v}$$
and βₑₓₜ^{v} the extended version of α^{v} and β^{v} through the day in five-minute intervals; and
where the cost function (J) is:
$$J = {\sum }_{j = 1}^{{N}_{days}} 2 \cdot {LBGI}_{j} + {HBGI}_{j} + {N}_{j , HT}$$
with N_{days} the number of days considered for the optimization (≤14 days), Nur the number of hypoglycemic treatments per day, and LBGIⱼ and HBGIⱼ the hypoglycemia and hyperglycemia indices per day.

88. The non-transitory computer readable medium of claim 65, wherein the historical data further includes a finite time range and basal and bolus modulators.

89. The non-transitory computer readable medium of claim 65, wherein the said simulation is based on custom modulation factors.

90. The non-transitory computer readable medium of claim 89, wherein said custom modulation factors are determined by parsing received historical data into finite time ranges with associated basal and bolus modulators based on said personalized model of the user's glucose metabolism.

91. The non-transitory computer readable medium of claim 89, wherein said custom modulation factors are defined as Pfactors.

92. The non-transitory computer readable medium of claim 91, wherein the said simulation is based on combining optimal modulation factors with said Pfactors to define final modulation profiles for at least one of: basal rate, carbohydrate ratio, and correction factor.

93. The non-transitory computer readable medium of claim 65, wherein said simulation features changes or non-changes to at least one of: basal rate profile, carbohydrate ratio profile, correction factor profiles, or user-specific glucose charts.

94. The non-transitory computer readable medium of claim 65, wherein said treatment includes changes or non-changes to basal and bolus settings.

95. The non-transitory computer readable medium of claim 65, wherein execution of the program instructions by said one or more computer processors cause the one or more processors to:
present the time spent in different glucose ranges via commonly accepted glucose charts.

96. The non-transitory computer readable medium of claim 65, wherein at least part of the historical data is obtained from one or more of the following:
an insulin pump device, continuous glucose monitoring device, a manual insulin injection device, a meal-tracking device, and an activity-tracking device.

97. The non-transitory computer readable medium of claim 65, wherein the insulin-dosing parameters are recommended to the user or automatically implemented by a closed-loop control or continuous subcutaneous insulin delivery system.

98. The non-transitory computer readable medium of claim 65, wherein execution of the program instructions by said one or more computer processors cause the one or more processors to perform one or more of the following:
record said plurality of actionable information and risk assessments to a database;
record said plurality of actionable information and risk assessments to a database accessible by the user;
present said plurality of actionable information and risk assessments to the patient via a display system;
present said plurality of actionable information and risk assessments to the user via a website or web application;
record said insulin-dosing parameters based on the personalized model of the user's glucose metabolism to a database;
record said insulin-dosing parameters based on the personalized model of the user's glucose metabolism to a database accessible by the user;
present said insulin-dosing parameters based on the personalized model of the user's glucose metabolism via a display system; and
present said insulin-dosing parameters based on the personalized model of the user's glucose metabolism via a website or web application.
